(19) United States
(12) Patent Application Publication
(10) Pub. No.: US 2016/0113503 A1
(43) Pub. Date: Apr. 28, 2016

(54) RAPID RATE-ESTIMATION FOR CELL PHONES, SMART WATCHES, OCCUPANCY, AND WEARABLES

(71) Applicant: David A. Benaron, Portola Valley, CA (US)

(72) Inventor: David A. Benaron, Portola Valley, CA (US)

(73) Assignee: AliphCom, San Francisco, CA (US)

(21) Appl. No.: 14/864,860

(22) Filed: Sep. 25, 2015

Related U.S. Application Data
(63) Continuation of application No. 14/864,857, filed on Sep. 24, 2015.
Application No. 14/864,857 is a continuation of application No. 14/555,377, filed on Nov. 26, 2014.
Application No. 14/555,377 is a continuation of application No. 14/552,468, filed on Nov. 24, 2014.
Application No. 14/552,468 is a continuation of application No. 14/552,690, filed on Nov. 25, 2014.
Application No. 14/552,690 is a continuation of application No. 14/554,053, filed on Nov. 26, 2014.
Application No. 14/864,857 is a continuation of application No. 14/555,059, filed on Nov. 26, 2014.
Application No. 14/555,059 is a continuation of application No. 14/555,554, filed on Nov. 26, 2014.
(60) Provisional application No. 62/054,873, filed on Sep. 24, 2014.
(60) Provisional application No. 62/053,780, filed on Sep. 22, 2014.
(60) Provisional application No. 62/050,828, filed on Sep. 16, 2014.
(60) Provisional application No. 62/050,952, filed on Sep. 16, 2014.
(60) Provisional application No. 62/050,900, filed on Sep. 16, 2014.
(60) Provisional application No. 61/989,140, filed on May 6, 2014.
(60) Provisional application No. 61/970,667, filed on Mar. 26, 2014.
(60) Provisional application No. 61/908,926, filed on Nov. 26, 2013.
(60) Provisional application No. 62/053,780, filed on Sep. 22, 2014.
(60) Provisional application No. 62/050,828, filed on Sep. 16, 2014.
(60) Provisional application No. 62/050,952, filed on Sep. 16, 2014.
(60) Provisional application No. 62/050,900, filed on Sep. 16, 2014.
(60) Provisional application No. 61/989,140, filed on May 6, 2014.
(60) Provisional application No. 61/970,667, filed on Mar. 26, 2014.
(60) Provisional application No. 61/908,926, filed on Nov. 26, 2013.
(60) Provisional application No. 61/908,926, filed on Nov. 26, 2013.
(60) Provisional application No. 61/970,667, filed on Mar. 26, 2014.
(60) Provisional application No. 61/989,140, filed on May 6, 2014.
(60) Provisional application No. 62/050,828, filed on Sep. 16, 2014.
(60) Provisional application No. 62/050,900, filed on Sep. 16, 2014.
(60) Provisional application No. 62/053,780, filed on Sep. 22, 2014.
(60) Provisional application No. 61/908,926, filed on Nov. 26, 2013.
(60) Provisional application No. 61/970,667, filed on Mar. 26, 2014.
(60) Provisional application No. 61/989,140, filed on May 6, 2014.
(60) Provisional application No. 62/050,828, filed on Sep. 16, 2014.
(60) Provisional application No. 62/050,900, filed on Sep. 16, 2014.
(60) Provisional application No. 62/053,780, filed on Sep. 22, 2014.
(60) Provisional application No. 61/908,926, filed on Nov. 26, 2013.
(60) Provisional application No. 61/970,667, filed on Mar. 26, 2014.
(60) Provisional application No. 61/989,140, filed on May 6, 2014.
(60) Provisional application No. 62/050,828, filed on Sep. 16, 2014.
(60) Provisional application No. 62/050,900, filed on Sep. 16, 2014.
(60) Provisional application No. 62/053,780, filed on Sep. 22, 2014.
(60) Provisional application No. 61/908,926, filed on Nov. 26, 2013.
(60) Provisional application No. 61/970,667, filed on Mar. 26, 2014.
(60) Provisional application No. 61/989,140, filed on May 6, 2014.
(60) Provisional application No. 62/050,828, filed on Sep. 16, 2014.
(60) Provisional application No. 62/050,900, filed on Sep. 16, 2014.
(60) Provisional application No. 62/053,780, filed on Sep. 22, 2014.

(51) Int. Cl. A61B 5/00; A61B 5/0205

(52) U.S. Cl. CPC A61B 5/0059; A61B 5/725; A61B 5/0205; A61B 5/7278; A61B 5/6898; A61B 5/6804; A61B 5/6803; A61B 2560/0247; A61B 2562/0238; A61B 2576/00

(57) ABSTRACT

Techniques for respiratory and metabolic monitoring in mobile devices, wearable computing, security, illumination, photography, and other applications may use a phosphor-coated broadband white LED to produce broadband light, which may be transmitted along with ambient light to a target (e.g., ear, face, wrist, or the like). Some scattered light returning from a target may be passed through a spectral filter to produce multiple detector regions sensitive to a different waveband and/or wavelength range, and the detected light may be analyzed to determine a measure of a respiratory rate or effort. In the absence of LED light, ambient light may be sufficient illumination for analysis. The disclosed techniques may provide identifying features of type or status of a tissue target (e.g., respiratory rate, heart rate, heart rate variability, heart function, lung function, fat content, hydration status, confirmation of living tissue, and the like).

### CROSS-REFERENCE TO RELATED APPLICATIONS

This application is a U.S. non-provisional patent application that claims the benefit of U.S. Provisional Patent Application No. 62/054,873, filed Sep. 24, 2014, entitled “Rapid Rate-Estimation Method for Cell Phones, Smart Watches, Occupancy, and Wearables,” which is herein incorporated by reference for all purposes; This application is also a continuation of co-pending U.S. patent application Ser. No. 14/555,377 filed Nov. 26, 2014, which claims the benefit of U.S. Provisional Patent Application No. 62/053,780 filed Sep. 22, 2014, U.S. Provisional Patent Application No. 62/050,828 filed Sep. 16, 2014, U.S. Provisional Patent Application No. 62/050,952 filed Sep. 16, 2014, U.S. Provisional Patent Application No. 62/050,900 filed Sep. 16, 2014, U.S. Provisional Patent Application No. 61/989,140 filed May 6, 2014, U.S. Provisional Patent Application No. 61/970,667 filed Mar. 26, 2014, and U.S. Provisional Patent Application No. 61/908,926 filed Nov. 26, 2013, ” all of which are herein incorporated by reference for all purposes; This application is also a continuation of co-pending U.S. patent application Ser. No. 14/552,468 filed Nov. 24, 2014, which claims the benefit of U.S. Provisional Patent Application No. 62/053,780 filed Sep. 22, 2014, U.S. Provisional Patent Application No. 62/050,828 filed Sep. 16, 2014, U.S. Provisional Patent Application No. 62/050,952 filed Sep. 16, 2014, U.S. Provisional Patent Application No. 62/050,900 filed Sep. 16, 2014, U.S. Provisional Patent Application No. 61/989,140 filed May 6, 2014, U.S. Provisional Patent Application No. 61/970,667 filed Mar. 26, 2014, and U.S. Provisional Patent Application No. 61/908,926 filed Nov. 26, 2013, ” all of which are herein incorporated by reference for all purposes; This application is also a continuation of co-pending U.S. patent application Ser. No. 14/552,690 filed Nov. 25, 2014, which claims the benefit of U.S. Provisional Patent Application No. 62/053,780 filed Sep. 22, 2014, U.S. Provisional Patent Application No. 62/050,828 filed Sep. 16, 2014, U.S. Provisional Patent Application No. 62/050,952 filed Sep. 16, 2014, U.S. Provisional Patent Application No. 62/050,900 filed Sep. 16, 2014, U.S. Provisional Patent Application No. 61/989,140 filed May 6, 2014, U.S. Provisional Patent Application No. 61/970,667 filed Mar. 26, 2014, and U.S. Provisional Patent Application No. 61/908,926 filed Nov. 26, 2013, ” all of which are herein incorporated by reference for all purposes; This application is also a continuation of co-pending U.S. patent application Ser. No. 14/554,053 filed Nov. 26, 2014, which claims the benefit of U.S. Provisional Patent Application No. 62/053,780 filed Sep. 22, 2014, U.S. Provisional Patent Application No. 62/050,828 filed Sep. 16, 2014, U.S. Provisional Patent Application No. 62/050,952 filed Sep. 16, 2014, U.S. Provisional Patent Application No. 62/050,900 filed Sep. 16, 2014, U.S. Provisional Patent Application No. 61/989,140 filed May 6, 2014, U.S. Provisional Patent Application No. 61/970,667 filed Mar. 26, 2014, and U.S. Provisional Patent Application No. 61/908,926 filed Nov. 26, 2013, ” all of which are herein incorporated by reference for all purposes; This application is also a continuation of co-pending U.S. patent application Ser. No. 14/555,059 filed Nov. 26, 2014, which claims the benefit of U.S. Provisional Patent Application No. 62/053,780 filed Sep. 22, 2014, U.S. Provisional Patent Application No. 62/050,828 filed Sep. 16, 2014, U.S. Provisional Patent Application No. 62/050,952 filed Sep. 16, 2014, U.S. Provisional Patent Application No. 62/050,900 filed Sep. 16, 2014, U.S. Provisional Patent Application No. 61/989,140 filed May 6, 2014, U.S. Provisional Patent Application No. 61/970,667 filed Mar. 26, 2014, and U.S. Provisional Patent Application No. 61/908,926 filed Nov. 26, 2013, ” all of which are herein incorporated by reference for all purposes; and This application is also a continuation of co-pending U.S. patent application Ser. No. 14/555,554 filed Nov. 26, 2014, which claims the benefit of U.S. Provisional Patent Application No. 62/053,780 filed Sep. 22, 2014, U.S. Provisional Patent Application No. 62/050,828 filed Sep. 16, 2014, U.S. Provisional Patent Application No. 62/050,952 filed Sep. 16, 2014, U.S. Provisional Patent Application No. 62/050,900 filed Sep. 16, 2014, U.S. Provisional Patent Application No. 61/989,140 filed May 6, 2014, U.S. Provisional Patent Application No. 61/970,667 filed Mar. 26, 2014, and U.S. Provisional Patent Application No. 61/908,926 filed Nov. 26, 2013,” all of which are herein incorporated by reference for all purposes.

### FIELD

The present invention relates generally to mobile and wearable computing used for biological parameter measurements. More specifically, low power consuming techniques for rapidly extracting heart rate, respiratory rate, and other features from a signal using a wearable computing device are described. Further, the described techniques include a low-power method for fitting the shape or timing between repetitive events to determine a heart or respiratory rate in a living subject, such as using intervals, wavelets, or other features to determine or estimate a heart or respiratory rate in a low number of cycles (e.g., two cycles, two heart beats, and the like, or less), resulting in improved user satisfaction, longer battery life, and more continuous measurements over time. Enabling systems and devices for incorporating or practicing the improved content-aware sensor are also disclosed.

### BACKGROUND

Conventional techniques for determining heart rate or other cycles involve simple counting. In conventional techniques, a nurse or physician holds the wrist or places fingers on the neck for heart rate, or listens to or watches movement of the chest or abdomen for respiratory rate, and counts. In order to get a good count, many cycles must be measured (error being a function most simply related to the square root of the count number), such as counting over 15 or 20 seconds for pulse, and 30 seconds to 1 minute for respiratory. Conventional techniques are inadequate for subjects such as runners by extending a period of time over which a count is performed, which can make a user impatient, waste battery life, result in an inadequate counting period for good accuracy, and generally decrease the commercial and personal value of a device implementing these types of conventional techniques.

Conventionally, noninvasive sensors for detecting heart rate or respiratory rate in an ambulatory subject also depend upon a counted signal; derived repeatedly in response to continuous illumination by a dedicated light source. This results in increased power consumption in both the illumination and the detection, and uses power to run the detection and processing amplifiers and circuits. Thus, power consumption increases whenever counting requires extended time. Conventional optical sensors that use continuous heart rate monitoring typically have high current drains. Further, some conventional devices for estimating calories use accelerometers. These devices estimate a calorie consumption using baseline calculations (such as Basal Metabolic Rate, or BMR) from age, weight, height, or other biometrics, and augment those using additional calories based on movement. These devices do not incorporate noninvasive and/or noncontact measures of respiration and, when moving part of a subject's body (e.g., while riding a stationary cycle) conventional devices typically underestimate caloric burn rates providing inaccurate results to users.

Thus, what is needed is a solution for real-time sensing without the limitations of conventional techniques.

### BRIEF DESCRIPTION OF THE DRAWINGS

FIG. 1 is a schematic of an exemplary operating system using a small multispectral filter;

FIG. 2A shows an exemplary fiber bundle multispectral filter;

FIG. 2B shows a photograph of an exemplary fiber bundle filter during testing;

FIG. 2C shows an exemplary sensor chip using spectral coatings on glass placed on a silicon detector chip, with collimating tubes and filter and shaping optics over each detector;

FIG. 2D shows a photograph of an exemplary sensor board built using coated spectral filters placed on silicon chip detectors;

FIG. 2E shows an exemplary schematic of a single-chip bio-aware sensor chip;

FIG. 3A shows an exemplary broadband LED constructed from individual LEDs for use in the infrared;

FIG. 3B shows a photograph of an exemplary broadband infrared LED source array;

FIG. 4 shows an exemplary optical spectrum measured from a broadband infrared LED;

FIG. 5A shows an exemplary real-time, non-contact heart rate data stream, collected in this case 3-5 times a second from multispectral sensor in a cell phone;

FIG. 5B shows a real-time, non-contact heart rate data stream, collected from a multispectral sensor focusing on blood in the arterial supply as compared to a chest-lead medical EKG.

FIG. 6A shows data spectral data from a hand collected from an exemplary spectrally resolved senor configured as a smart proximity detector to detect tissue;

FIG. 6B shows data spectral data from an arm with a sleeve covering the wrist collected from an exemplary spectrally resolved senor configured as a smart proximity detector to detect tissue;

FIG. 7 shows data from an exemplary wrist-based based sensor during exercise showing heart performance;

FIG. 8A shows a schematic side-view of a system incorporating an exemplary sensor into a loose-fit wrist-band;

FIG. 8B shows a schematic view of a system incorporating an exemplary sensor into a wrist watch;

FIG. 8C shows a system incorporating an exemplary sensor into a loose-fit non-contact pendant;

FIG. 8D shows an exemplary system incorporating the sensor into wearable glasses;

FIG. 8E shows an exemplary system incorporating the sensor into an energy-saving motion sensor for illumination control;

FIG. 8F shows an exemplary system incorporating the sensor into clothing;

FIG. 8G shows an exemplary system incorporating the sensor into an earphone earbud;

FIG. 9A shows an exemplary recessed non-contract sensor with the illumination and detection on the same chip;

FIG. 9B shows an exemplary non-contact recessed sensor where the white LED illuminator is separate from the detector;

FIG. 10A-B show exemplary respiratory rate detected using the arterial signal size. FIG. 10A shows loose fit oxy- and deoxy-hemoglobin data measured during exercise from a human subject over 100 seconds, with a filter with a time constant of 0.15 seconds, emphasizing the arterial pulse variations. FIG. 10B shows the same data with a 2 second time constant, emphasizing the arterial respiratory variations;

FIG. 11 shows an exemplary schematic algorithm;

FIG. 12A-B shows data analyzed for oxygenation in accordance with an exemplary schematic algorithm, and compartmentalized into venous and arterial compartments after both stabilization for skin changes, and differential analysis to emphasize changes over time. FIG. 12A shows calculations for changes in oxy- and deoxy-hemoglobin. FIG. 12B shows calculations resolved just to the arterial pulse compartment;

FIG. 13 shows exemplary techniques for using intervals to determine rate, in this case heart beat interval accuracy as determined by arterial compartment pulse and by EKG from data during exercise and movement, with a correlation coefficient of 0.94;

FIG. 14A-B show model data of how exemplary interval-based and counting-based rate estimation differ;

FIG. 15 shows a plot of respiratory rate as measured and determined in accordance with the described techniques on a human subject breathing at a controlled rate;

FIG. 16 shows cumulative calories expended as measured and calculated in accordance with exemplary techniques on a human subject under study conditions;

FIG. 17 shows an exemplary multispectral signal detected using ambient light; and

FIG. 18 is a block diagram illustrating an exemplary computer system suitable for rapid rate respiration determination.

### DETAILED DESCRIPTION

Various embodiments or examples may be implemented in numerous ways, including as a system, a process, a method, an apparatus, a circuit, a device, an article of manufacture, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

For purposes of describing various examples, the following terms are described generally, but are not necessarily limiting to the techniques described. One of ordinary skill in the art may understand that said descriptions are provided for purposes of example only and that these descriptions may be expanded, contracted, or modified beyond the scope of the descriptions that follow.

Ambient Light: In some examples, ambient light may refer to light present in an environment. Ambient light is often broadband, that is available over a wide range of wavelengths to perform a detection or analysis, for example by solution of multiple simultaneous spectroscopic equations using a set of optical filters over a sensor. For some content-aware sensors, a broadband range of at least 100 nm can at times be sufficient, while an exemplary bio-aware embodiment using sunlight in the environment having wavelengths covering over 300 nm or more from 440 to 740 nm. If water and fat detection are added, which can advantageously use peaks for water at 960 nm, and for fat around 920 nm, then a range of 440 nm to 1000 nm may be used. Room illumination often appears white to the eye, and is also often broadband.

Hydration Status: In some examples, the overall water and fluid balance of an individual. In the simplest view, hydration reflects whether an individual has sufficient, insufficient, or excess body water. In other examples, more complex analysis can look at which body compartments have water (such as intravascular fluids, extracellular fluids such as tissue edema, intracellular fluids).

Reduced-Power: In some examples, power consumption lowered as compared to similar sensors through the use of ambient light as a light source for some or all of sensor detection.

Respiratory Rate: In some examples, the rate at which breathing occurs. Breaths may be effective, ineffective (such as during obstruction), or even absent (such as in coma, or during certain types of sleep apnea). As an example, standard measures known to those skilled in the art, including breath volumes (tidal volumes), and the amount of air moved each minute (minute volume) may be used.

Content-blind: In some examples, a gesture or event sensing approach that is dependent on a physical act or movement, but is insensitive to state, type, identity, or condition of the gesturer (subject) or object. For example, pressing a key is content-blind, as it does not matter if it is a pencil, a dead cat's paw, a monkey with a banana, or a user's finger that places physical pressure upon the keys or icon. Regarding keyboards (e.g., smart phone, computer, or the like), in some examples, physical pressure of an object pressing a key (or for gesture sensitive devices, the movement of the touching object) is important, not the identity of the object doing the actuating.

Content-aware: In contrast to content-blind sensing, a sensing approach or system in which the sensor is able to intelligently detect and extract certain features about the person or object triggering the sensing event. For example, to analyze and detect that a hemoglobin-containing living hand or a chlorophyll-containing leaf appears in a photographic image are content-aware determinations. Content-awareness allows, for example, a proximity sensor to recognize that an object near a sensor is a living hand or finger, rather than a sleeve or a book, for specific gesture recognition with reduced error. This is not merely a pressure based or touch based system, such as a grip pressure sensor, but an actual spectral analysis to determine the type or state of the target. Similarly, the color correction of a photograph can be improved if an image sensor is able to determine that a certain feature is human skin, or that another feature is sky, based on a spectral analysis of (or in additional to using traditional image processing of) the spectral information obtained by the sensor.

Bio-aware: A content-awareness that detects features of a live user, such as the presence of hemoglobin, a heart rate, a body metabolism, a specific body composition, or recognition that an object near a sensor is a hand or finger for body-specific gesture recognition. A camera that color corrects pixels, or counts living objects present, based on the detection of hemoglobin in the one or more pixels, is bio-aware. This again is more than mere physical detection (such as a facial recognition algorithm using the shape of eyes and mouth) that would be fooled by a color photograph. A bio-aware method determines formal content such as chemical composition, not just physical appearance.

Filter: A device that restricts incoming light to of a specific type of light, such as by wavelength range, polarization, or other optical feature.

Spectral Filter: A filter that specifically restricts incoming light based on color or wavelength, usually restricting it to a specific set of colors or range or wavelengths. For example, an interference coating that more or less allows wavelengths from 550 to 560 nm to pass is a 10 nm bandwidth spectral filter.

Sample or Target Sample: Material illuminated then detected by a sensor for bio-aware spectrally resolved analysis. A target sample may be an object, or can be living tissue.

Target Indicator: An optical characteristic specific to the target being measured.

Scattering: The redirection of light by a target sample. Most biological tissues scatter light, which is typically why we can see or detect them from light that scatters back from living tissues onto our retinae.

Light: Electromagnetic radiation from ultraviolet to infrared, namely with wavelengths between 10 nm and 100 microns, but especially those wavelengths between 200 nm and 2 microns, and more particularly those wavelengths between 550 and 1900 nm where chemical bands appear that allow unique identification.

Broadband Light: Light produced over a wide range of wavelengths to perform a detection or analysis, for example by solution of multiple simultaneous spectroscopic equations using a set of optical filters over a sensor. For some content-aware sensors, a range of at least 100 nm can at times be sufficient, while an exemplary bio-aware embodiment uses a white LED that produces light over 300 nm or more from 440 to 740 nm.

Light Source: A source of illuminating photons. A light source can be external, such as sunlight.

LED: A light emitting diode.

White LED: A broadband, visible wavelength LED, often comprised of a blue LED and a broad-emitting blue-absorbing phosphor that emits over a wide range of visible wavelengths. Other phosphors can be substituted, including Lumigen, as discussed herein. As used in the examples herein, any broadband LED could be used, even if not emitting over a full (white) spectrum. For example, an LED emitting over a range of 100 nm would be considered to be broadband.

Wearable: A sensor or device that can be worn on, in, or near the body, such as smart glasses, smart jewelry, or clothing with embedded sensors. The wearable can be an electronic device, like an earphone, an ocular implant or contact lens, a mouthpiece or tooth cover or replacement, or a monitoring band.

Motion: Movement, such as running during exercises.

Non-contact: A measurement in which the detector and/or the illuminator is not in contact with the tissue. This can be a short distance (such as a 2-10 mm spacing under a loose wristband) or a medium distance (such as a security and movement detector on the ceiling of an office room, or an occupancy sensor or counter used to control illumination power) or quite long distance (such as a glasses based sensor that overlays the heart and respiratory rate on people in your visual field even if both of you are in motion).

Loose Fit: A non-contact (or optionally non-contact) sensor or device configuration in which the data is collected without required contact with the tissue, such as a loose bracelet or a pendant.

Hemoglobin (or Heme): A pigmented molecule that carries oxygen in the blood. It is relevant to the exemplary techniques that hemoglobin comes in many forms. In humans the primary forms are oxyhemoglobin (heme with oxygen) and deoxyhemoglobin (heme without oxygen). The reddish color of arterial blood comes from oxyhemoglobin being the main pigment (arterial hemoglobin is often over 96% oxyhemoglobin and under 4% deoxyhemoglobin), while the bluish color of venous blood is from the presence of large amounts of deoxyhemoglobin (venous hemoglobin is often around 30% deoxyhemoglobin with 70% oxyhemoglobin).

Software: In some examples, software coded instructions may be implemented as software, firmware, circuitry, application program code, instruction sets, computer programs, applications, or the like for implementing the techniques described herein. Code may be stored on a non-transitory physical media, and are intended to direct a microcontroller, dedicated application-specific physical integrated circuit (ASIC), phone, fitness product, or other physical sensor systems to collect, analyze, and produce results from data collected from the sensors.

Measurement: A non-transient value determined over a period, or at one instant of time. A measurement is a stable form of information that can be stored in machine-readable hardware, such as a memory location, or can be used in mathematical equations or analysis. In some examples, memory may be implemented using various types of data storage technologies and standards, including, without limitation, read-only memory (“ROM”), random access memory (“RAM”), dynamic random access memory (“DRAM”), static random access memory (“SRAM”), static/dynamic random access memory (“SDRAM”), magnetic random access memory (“MRAM”), solid state, two and three-dimensional memories, Flash®, and others. Memory may also be implemented using one or more partitions that are configured for multiple types of data storage technologies to allow for non-modifiable (i.e., by a user) software to be installed (e.g., firmware installed on ROM) while also providing for storage of captured data and applications using, for example, RAM. Once captured and/or stored in memory, data may be subjected to various operations performed by other elements beyond those described herein.

In contrast, there can be ample signal in certain repetitive functions that can allow for a rapid estimate, with very few cycles. By using time intervals, rather than counting the signals, a rate can be determined in two cycles, or even less if additional physiology is considered. This allows a rapid lock-in, as well as a shortened on period in power sensitive devices. Resulting in more rapid fulfillment of a user query as well as a lower power requirements than may be used if a light source would need to be powered over a continuous period of monitoring.

A low power system is particularly useful for turning on or unlocking a phone in response to the presence of a hand or face, or for detecting the heart and respiratory rates of people within an image sensor's field of view. In contrast, there are methods that allow determination of a repetitive cycle in half to 2 cycles, or less. Such methods include monitoring cycle portions of known length relative to rate, or looking at cycle intervals, to rapidly determine a cardiac or respiratory rate.

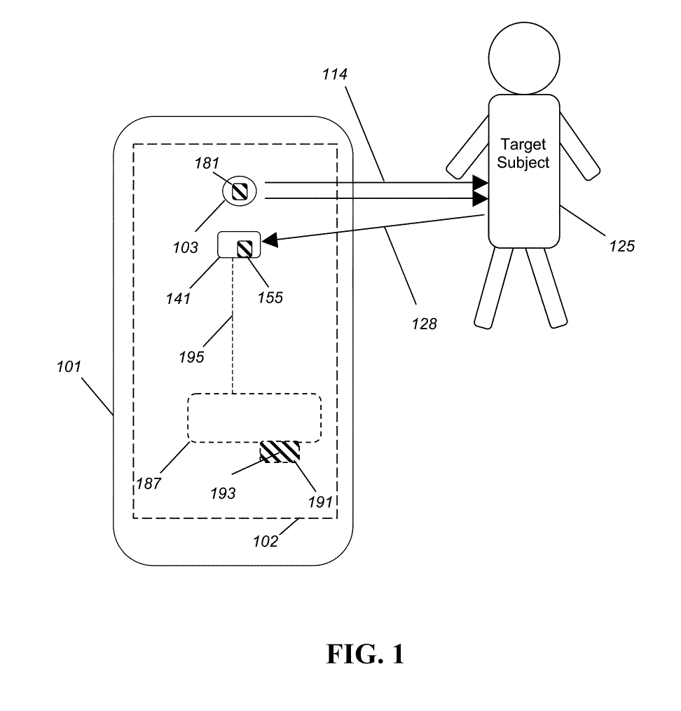

In some examples, a device source such as that shown in FIG. 1, smart phone 101 has illuminator 103 and image camera detector 141. Illuminator 103, detector 141, and the processing and control circuitry and software together form sensor 102.

Illuminator 103 is a white LED. Broadband white light is emitted forward, in a beam as shown by light path vectors 114, with some light reaching (and optically coupled to) target 125. Of note, target 125 is shown for illustrative purposes as a human subject, and is neither a part of the apparatus or system, nor is the human body or human subject claimed as patented material.

A portion of the light reaching target 125 is scattered and reflected, and returns as returning scattered and reflected light 128 into the smartphone camera image detector 141. Optionally, detector 141 could be a point detector, a linear array, or even one or more discrete detectors, provided that data representing filtered returning scattered light from the target sample is sensed and measured.

In this embodiment, detector 141 has added spectral filter 155. This filter allows light of a certain color range onto certain pixel elements of detector 141. In this case, filter 155 may cover a small region of the image sensor, so as not to interfere with image collection for other purposes, such as photographs. Filter 155 in this example has 7 filter ranges, each 5 nm FWHM wide, with center wavelengths at 525, 540, 555, 570, 585, 600, and 630 nm. Additional ranges may include 900, 920, 940, and 960 nm, and for these wavelengths in phones with white LED illumination, the 900-980 nm illumination may be generated from an IR source in the phone's illumination or from ambient or other illumination sources). Sensor 102 measures less than 3 mm in width. Filter 155 may incorporate a polarizing coating as part of its filtering function. Filter 155 is attached to detector 141 using optical epoxy.

The non-contact measurement can be enhanced using polarization filters, integrated into the emitter and at 90-degrees (cross-polarized) on the detector. This is because light that reflects off of the skin retains polarization, and can be blocked using a correctly positioned polarizer on the detector (in this case cross-polarized, but it may be a different angle in other situations). In contrast, light entering the tissue is depolarized during multiple scattering, and thus travels in greater percentages through the cross-polarizer on return, thus enhancing the light. In studies, we found that the apparent hemoglobin (a measure of travel through tissue) was up to 2-fold higher when crossed-polarizers were used. These are shown in FIG. 1 as a polarizer layer included as part of the construction of filter 155, and optional polarizer 181 over illuminator 103.

Next, some or all of the data from image detector 141, including the filtered pixels, is read and processed by embedded microcontroller 187 (such as those typically present to operate cell phones, and shown dashed as it is located internally as part of the cell phone main circuitry) based on machine-readable code 193 saved on physical medal, such as ROM, RAM, or flash disk physical memory 191, or other types of memory technologies, which may be connected over electrical connection 195.

The machine readable code may optionally be system software saved as a machine-readable code embedded within a non-transitory physical memory ROM, or it could be an “app” (a downloadable code available for installation and/or purchase and then stored within a non-transistory physical memory), or it could be an “API” (an installed driver for a specific sensor, such as would be provided by a manufacturer with a given physical sensor set and using instructions stored on non-transitory computer readable media).

The precise design of software 172 may depend on the smartphone, watch, earbud, anklet, camera, or bracelet processor, but its function is to process the image and provide raw or processed results to the device or system For example, one result would be the photon counts for each of the filtered region, with each filter region covering multiple image pixels. Another result could be a processed result, in which least-squares fitting is performed against a spectral standard in order to determine the presence of hemoglobin in the image. Another result could be that the measurement is processed over time in order to produce a heart rate estimate. In some examples the returning light may be processed for type, state, identify, or gesture, and the broadband white LED source may be used for illumination.

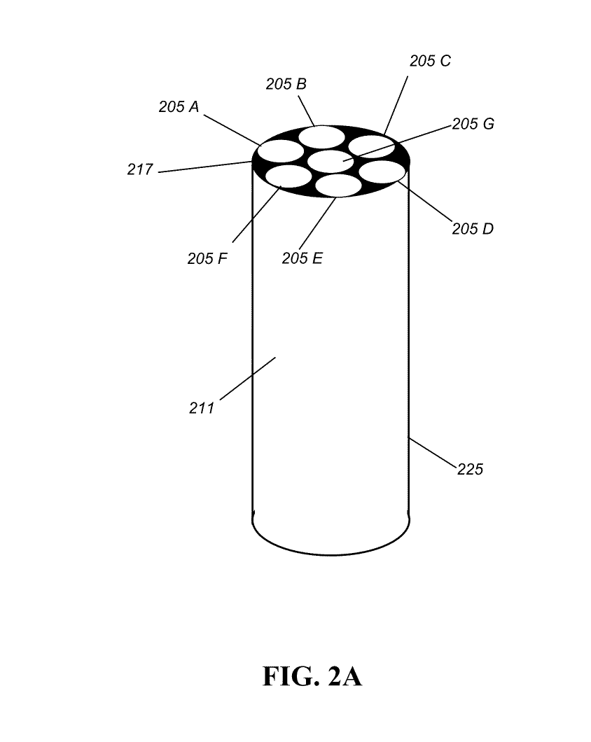

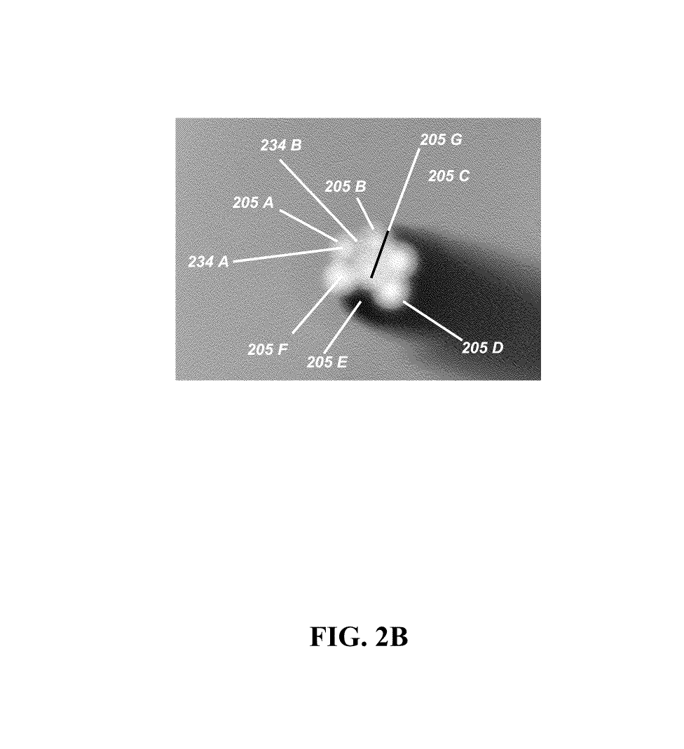

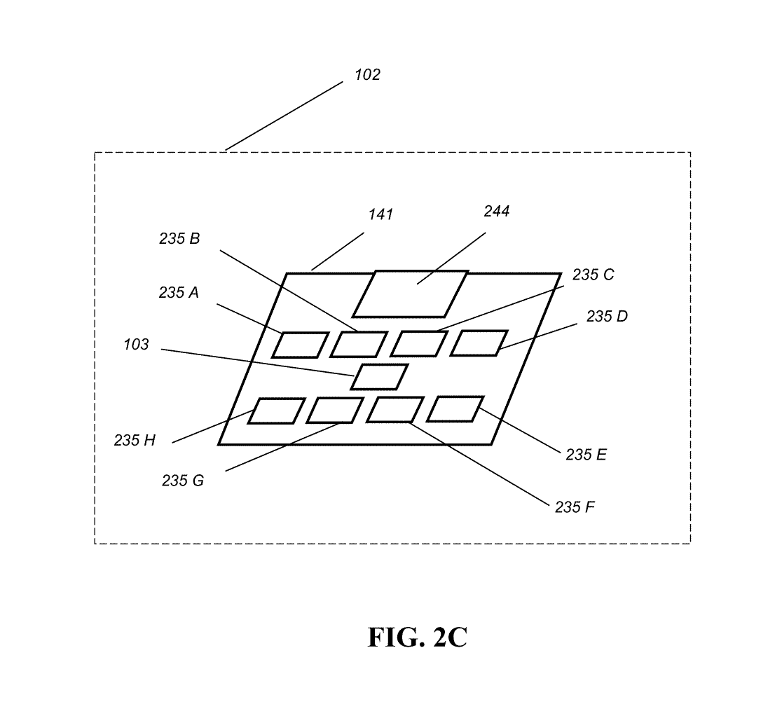

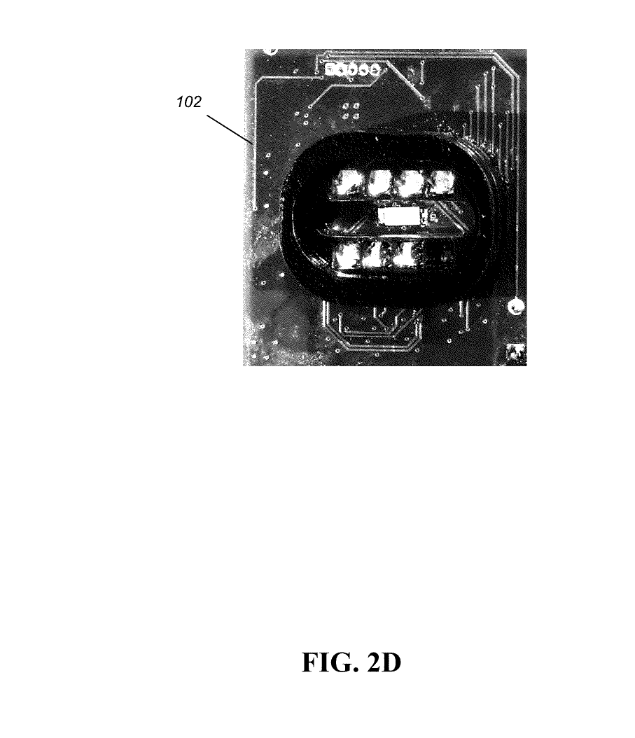

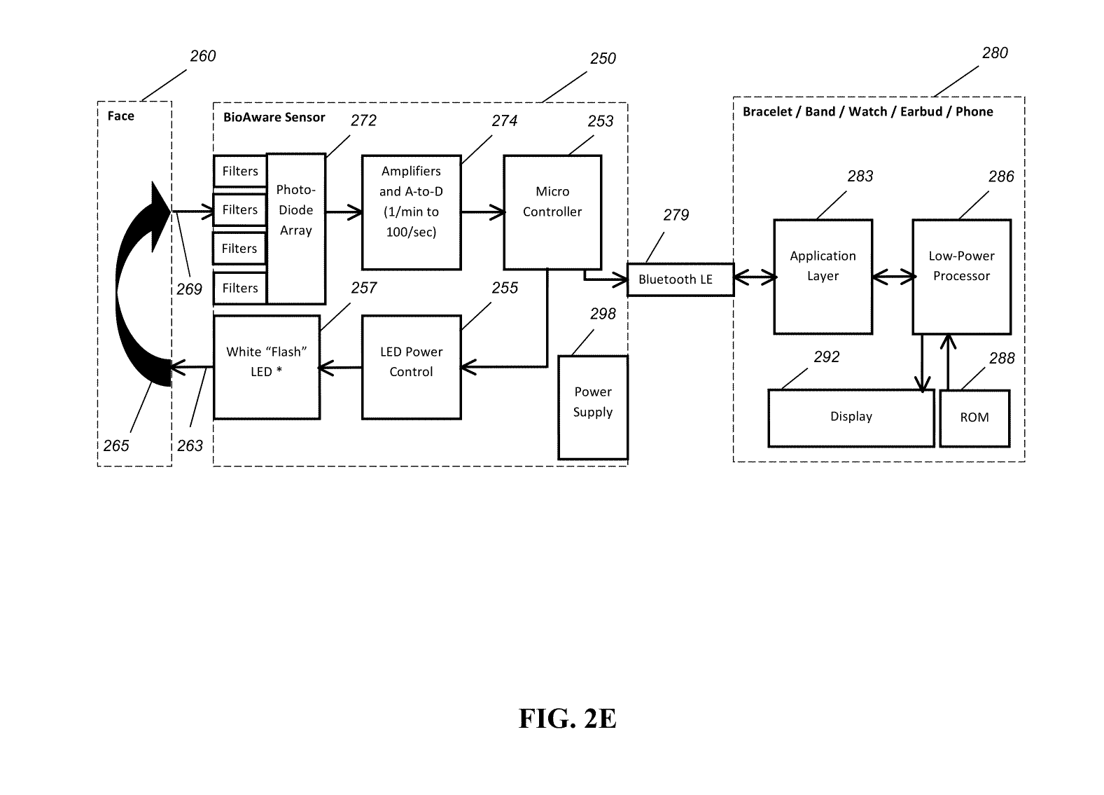

Spectral filter 155 of this preferred embodiment is now briefly described, as shown in FIG. 2. Here, filter 155 is shown as all of FIG. 2A and composed of 7 optical fibers 205A through 205G (one or more wavelengths described in Example 1 are omitted for clarity). The number of fibers can vary, even down to 1 but more typically 3 to 12, depending on application. Each of the fibers has a spectral filter coated onto the top end of each fiber, and the filter differs for each fiber 205A through 205G. In this example, the fibers are arranged in a circle of 6 outer fibers, with one central fiber. Alternatively, the fibers can be in various layouts, including different shaped patterns (square, linear row, star). In the construction of this custom filter, the fibers are first provided a filter coating, with each wavelength range run in a separate deposition chamber using pre-cut pre-polished (or cut) fibers, with thousands or more in each deposition chamber run. Then, one fiber of each wavelength is taken, prepared with epoxy on the side of the fiber, and placed into glass tube 211. Alternatively, tube 211 could be plastic, epoxy, resin, metal, or other material, provided it allows alignment and securing of the fibers. Once the black epoxy, shown as black epoxy 217, hardens, then distal end 225 can be polished.

A photograph of an actual 7-fiber system we constructed is shown in FIG. 2B, where all fibers (except fiber 205E) are illuminated. The image and localization improves with better polishing, spectral filter deposition, and other improvements to the fiber tip. This tip as shown in FIG. 2B can then be glued directly to detector 141 as shown in FIG. 1 as filter 155 on detector 141. The fibers are then attached (in this case, clear epoxy optical glue) to the face of the CCD for direct transfer of the transmitted photos to the image sensor detector.

Alternative constructions are optionally possible. For example, there may be more or fewer than 7 filter ranges, depending upon the intended application. Next, there may be more than 1 fiber for each wavelength range. For example, there may be 10 of each fiber, for a total of 700 fibers in the set. Then, after placement on the CCD, a calibration may need to be performed to assign each image sensor region to a pixel spectral range, allowing averaging and integration at several locations for each range.

Another alternative format for filter 155 as used in sensor 102 is shown as FIG. 2C. Here, the filter is comprised of a number of small filters assembled on one or more silicon detectors 141, shown as filters 235A through 235H, which are placed over the surface of detector(s) 141. Amplification of the signals occurs in integrating amplifiers, microcontrollers, and instructions stored in non-transient machine-readable physical memory 244.

A photograph of such a device as constructed and tested is shown in FIG. 2D, where custom optical filters 235A-D and 235F-H (Omega Optical, Brattleboro, Vt.) with collimating lenses can be seen on top of silicon photodiodes or phototransistors. Here elements are added above the silicon detectors to complete sensor chip 102, such as a collimating spacers, polarizers, and focusing lenses can be added, such as to reduce the angular bleed through of light into the spectral filters. The darker-appearing detector has a transparent region 235E in FIG. 2D, and no collimating lens, allowing unfocused and spectrally unfiltered white light to reach the detector).

A schematic of a sensor chip is shown in FIG. 2E. Here, sensor board 250 has microcontroller 253 (which can be an off-board controller) using LED power control 255 to power white LED 257 configured in flash mode. Light travels without tissue contact along light path 263 to a body part. As in FIG. 1, the human body and tissue are shown to provide an understanding of the operation of the described techniques. Light scatters through the tissue along light path 265, and then leaves the tissue along backscattered and remitted light path 269, re-encountering sensor chip 250 and entering filter and photosensor detector array 272.

As described, the spectral filters can be separate elements, one filter element tuned by angle of entry across a range, or filters deposited directly on the detector substrate. In this case, interference filters were on separate glass substrates (custom 3×3 mm filters, Omega Optical, Brattleboro, VT) ranging from 5 to 40 nm FWHM, and were glued on each photodiode detector using optical quality UV set glue. A polarizer and lens were additionally added to the stack above each filter. The detector may be CMOS, a photodiode, a phototransistor, or any number of suitable optical detectors known in the art. In this example, the detectors are 8 photodiodes (Vishay Temd 7000, or larger).

Detector array 272 creates an output measureable amplified and digitized by amplifier and A-to-D converter 274. In this case, the detector outputs are captured and integrated by low noise CMOS or BiFET amplifiers (analog devices AD823A), and translated to 16-bit digital sample/hold A-to-D converter (Linear LTC1867L). High gain channels reach 66% saturation at 16 uW/cm². The measurement can be improved by use of MOSFET amplifiers, and also by using higher-gain phototransistors, or even avalanche photodiodes (though the avalanche bias may increase the complexity of the chip and the cost of the sensor). Background estimation can be done by flashing the light at brief intervals. Each measurement filter channel is low pass filtered in two passive stages using a 1.2 ms time constant to control noise, and the light source itself is flashed on for 2 ms before a reading is taken. The system using less than 1 mm² of photodiode at each wavelength operates with 8 bit effective signal. By using a full 7.6 mm^{2 from a} 3×3 mm detector photodiode, 11 effective data bits can be obtained in this manner. For heart rate hemoglobin pulse signals, 8-14 bits is recommended.

As shown in shown in FIG. 2E, signals leave board 250 and are transmitted over link 279 to a bracelet, band, watch, earbud, phone, or other device. Link 279 can be an I2C wire, or even a Bluetooth connection (such as Bluetooth Low Energy, or Bluetooth LE). Sensor 102 may encompass both board 250 as well as device 280, either as a stand-alone sensor or as an embedded system within another system, device, wearable, article of clothing, camera, sensor, or other device. Here, device 280 includes machine-readable non-transient machine code stored in stable, readable ROM 288, and executed in this case as app layer 283 running on processor 286. Display 292 may provide results, feedback, warnings, or upload confirmations to a user. It may even display messages from a concerned physician who is responding to the data collected by sensor 102.

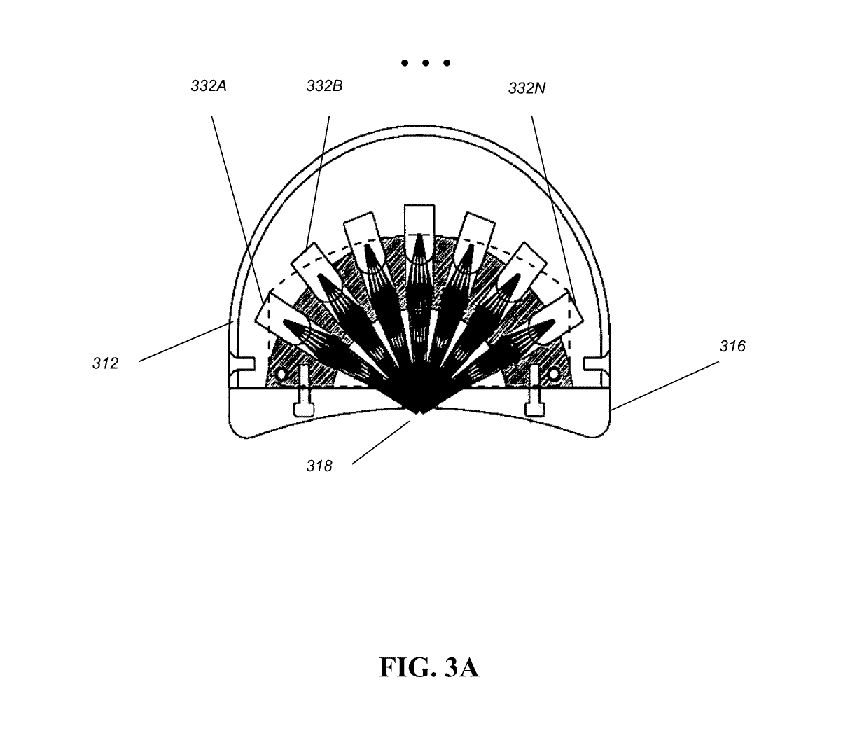

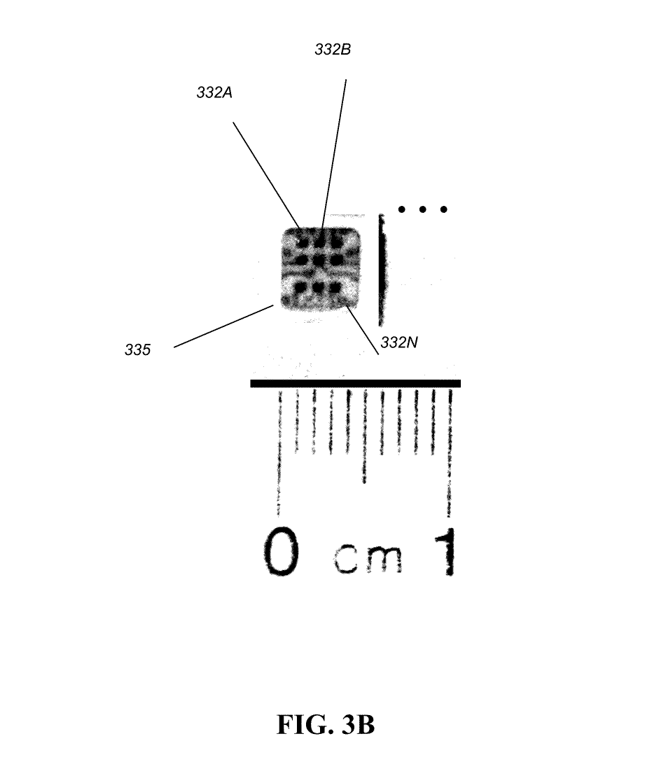

Alternative formats are also possible for the broadband light source instead of using a single white LED. One example is a multiple LED source, shown in FIG. 3A. Such a combined LED may be used when measuring, for example, fat and water using the spectral peaks in the 700-1000 nm range, a region not supplied by most conventional white LEDs found in cell phones and other mobile devices. Here, frame 312 with bottom 316 and opening 318 holds multiple LEDs 332A to 332N. These multiple LEDs, which can include broadband LEDs such as a white LED, are inserted into frame 312. Light from the multiple LEDs is focused or concentrated out exit opening 318, to provide broadband light.

When manufactured, the light source can be significantly more compact, as shown in the photograph in FIG. 3B. Here, multiple LEDs 332A through 332N are surface mount LEDs on PC board 335.

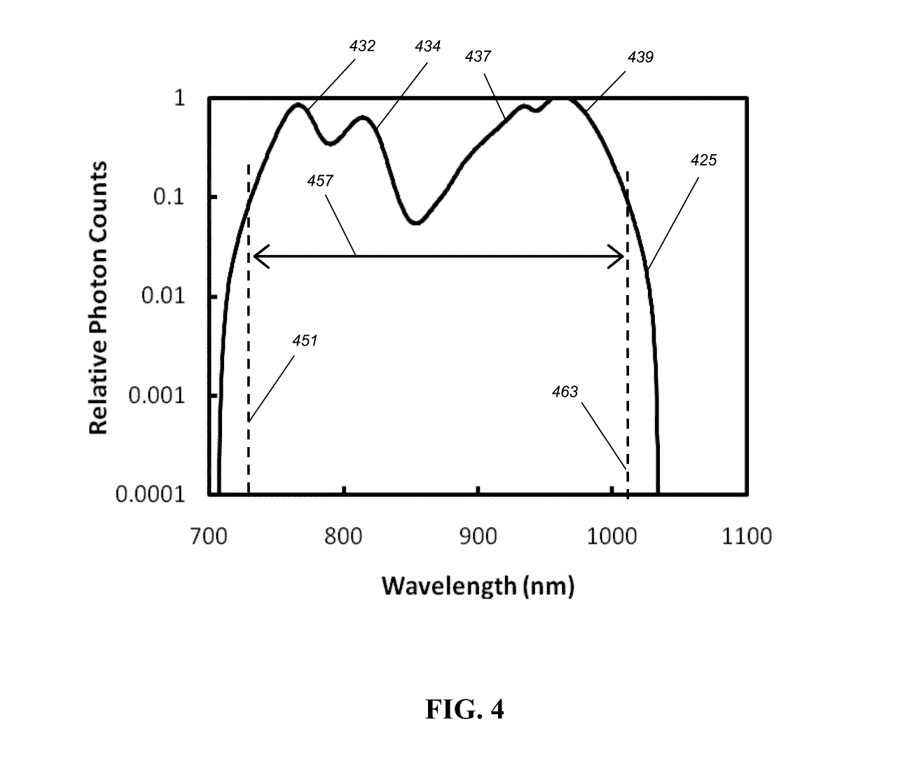

Light output from this multi-element light source is plotted in FIG. 4. Here, light emission is detected from about 700 nm to over 1000 nm, with light usable for over 300 nm of spectral width, from mark 451 to mark 463, with very little light by mark 425. Of note, the spectrum plotted shows peaks at peak 432, peak 434, peak 437, and peak 439, reflecting the peak contribution of certain LEDs used to build the light source. The width of the light output is shown as spectral width 457.

Smart phone 101 is turned on, and the spectral physiology app is selected by the user and started. For example, in an Android system, the app icon is located and touched, launching the app.

The app turns on phone white LED 103 and begins to collect data from camera detector 141. Data from detector 141 is accessed using software, in this case written in android language and compiled using the Android software development kit (SDK), available online (for example, at http://developer.android.com/sdk/index/html). Image data from detector 141 is available as RGB data (or as luminance and color, convertible to RGB using known equations). However, under spectral filter 155 the image from the lens is replaced by data from the fiber ends. An example of such data is shown in the image in FIG. 1, in this case collected using a USB plug in camera for a PC computer, dissembled and modified to have filter 155 attached and glued to the surface. The app software has already been calibrated to know which image pixels correspond to which filter, such as fiber center region 234A in FIG. 2B, and to ignore the overlap areas between fibers where two or more fibers overlap, such as fiber overlap region 234B in FIG. 2B. With many pixels of a detector covered by this filter, one may average the pixels to add statistical strength. What is produced by this combination is a table of the intensity at each of these wavelengths, which can then be analyzed in various ways.

This data may be collected on a spot basis for measurements without real-time change (such as water/fat composition), intermittently for values that change over minutes (such as cardiac performance), and nearly continuously (such as every 50 ms) for values such as heart rate, for which a continued change is key to extracting the value. These determinations are shown in more detail in the illustrative examples that follow.

In some examples, the breadth of uses of the described techniques is best understood by examples, provided below. These examples are by no means intended to be inclusive of all uses and applications of the apparatus, merely to serve as case studies by which a person, skilled in the art, can better appreciate the methods of utilizing, and the scope of, such a device.

### EXAMPLE 1

### Non-Contact Heart Rate Determination

In this example, illuminator 103 is a white LED embedded into a Samsung Galaxy S3 smartphone. Software app 172 is a custom software loaded into a machine-readable physical memory (4 Gb micro SD card, San Disk) placed into the external SD card slot of the Galaxy phone, and installed using the Android operating system (Android 4.4, Google) on the phone. The app is launched using the Android touch interface. Multiple filters allowed multiple bands wavelength bands to be collected.

Upon launch, Software app 172 turns on illuminator 103, as well as displays a camera image from detector 141, which shows a hand placed into the image sensor view, but not necessarily in contact with the sensor. A pixel region corresponding to sensor intensity averaged over 100 pixels for each of these spectral ranges every 300 milliseconds is captured.

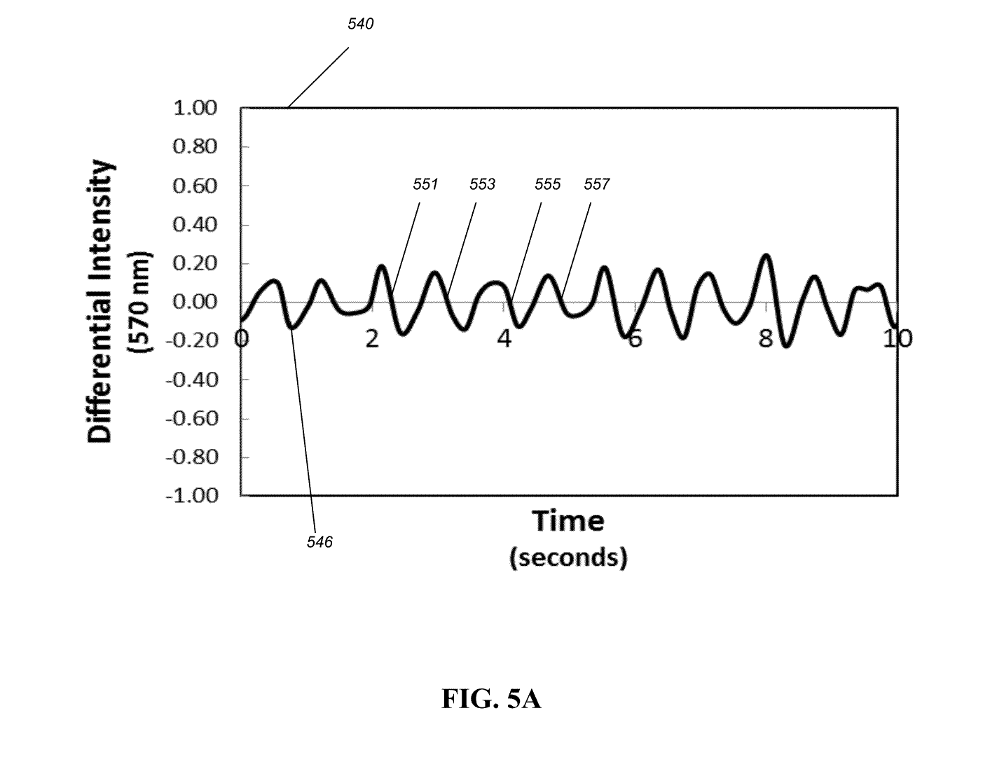

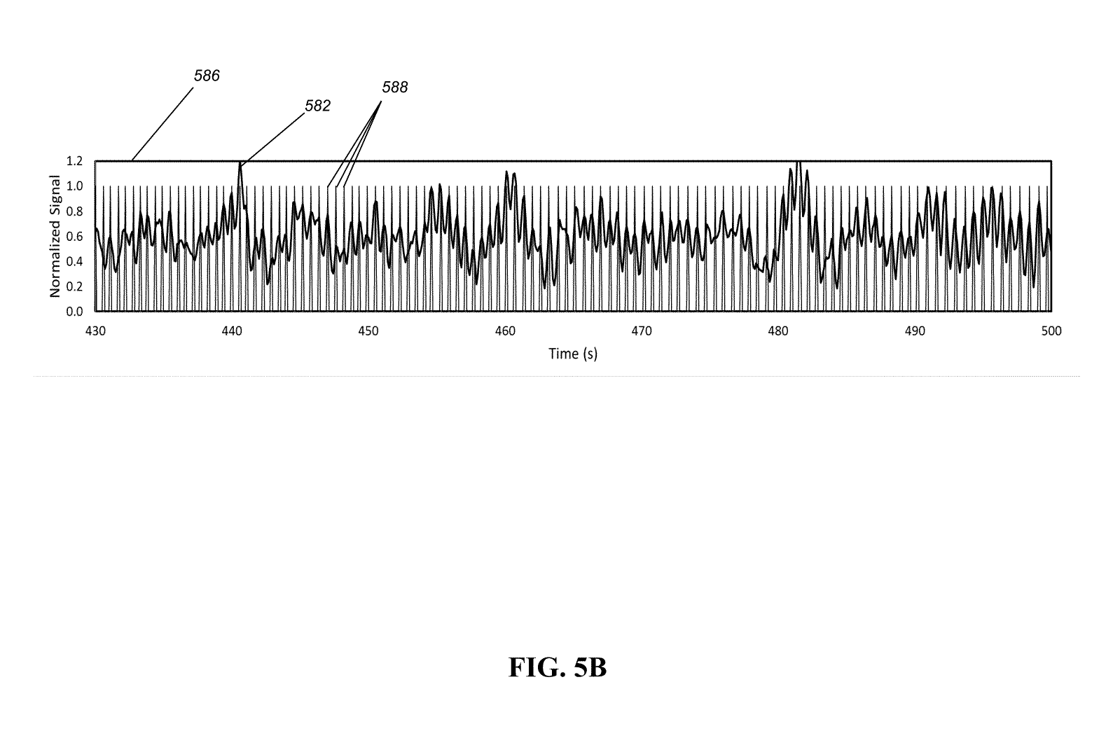

After capturing a spectral channel, the intensity is processed for change over time (a differential plot of intensity changes with respect to time). Here, the value is plotted versus time. The data are shown in FIG. 5A.

In FIG. 5, a time-varying output can be seen. In this case, the value of the output is determined as the normalized measurement from the 570 nm channel, minus a baseline change correction from a base-correction average of the measurement in the 460 and 630 nm channels. From this heart rate can be calculated simply by counting the peaks, using any of a number of methods familiar to those skilled in the art. One exemplary approach is to determine the beat-to-beat interval (i.e., the time between peaks). This allows for beats that are dropped to be detected as double-wide intervals which can be rejected, producing a more stable measurement in response to movement noise.

Alternatively, raw data, or interim determinations such as intensity changes over time, may optionally be displayed. Also, simply the changes in intensity at 570 nm (or other channels) may be plotted, as in a stable lighting environment the major change over intervals of seconds is the absorbance change caused by changes in hemoglobin.

For processing, a first differential (with respect to time) is determined, producing the varying measurement shown at plot 540 in FIG. 5A. Here, varying intensity 546 has peaks and troughs, which correspond to changes in hemoglobin volume with the pulsing of the heart. Peaks can be seen at 551, 553, 555, and 557. Each of these corresponds to one heartbeat. By determining a heart rate using the beat-to-beat intervals, and discarding the intervals with dropouts, a heart rate is determined; in this case, a heart rate of 2 beats/minute is measured and displayed.

Next, we constructed a research probe that allowed the sensor and broadband light source, of the types shown in FIG. 1, to be incorporated into a loose wristband system, with data collected at a multiple wavebands.

Rather than use other indirect measures, such as other fitness monitors, we have compared the performance of this wristband to a chest electrode EKG, to test accuracy. Data were recorded from a human volunteer during an exercise protocol, as described in the previous example. This subject also wore an accelerometer, a pulse oximeter, and several other instruments that monitored multiple functions during the study.

The heart rate signal, in some examples, in this case using 8 waveband multispectral data, is shown as plot line 582 in graph 586 of FIG. 5B. Also recorded at the same time, and plotted in FIG. 5B are the multiple, repetitive, narrow spikes of the QRS complex from a gold-standard chest lead EKG, shown as plot marks 588. The EKG records the electrical pulses from the heart with millisecond accuracy (when measured at 250 Hz with interpolation).

Comparing the signals visually at first, it can be seen by eye that there is a peak in the calculated heart rate signal with nearly every electrical signal, and very few such peaks visible where there is no EKG signal. This validates that the arterial signal has been extracted accurately, and that the timing of the signals is not invalidated by the EKG.

Instead of a visual assessment, another method of assessing the accuracy of these measures is to determine the interval between heartbeats, in milliseconds between beats or in effective heart rate at a given interval (e.g., an interval of 500 milliseconds corresponds to 120 beats/min), and compare these two measures. This beat-to-beat interval can be compared on a beat to beat basis, or averaged. In this following example, interval data were plotted as a running boxcar average over a moving 5 second window.

In some examples, measurement of the heart rate occurred during hard exercise, and would have been noisy or unreadable if using just one wavelength. In order to perform this calculation, multiple wavelengths were used to correct for movement artifact, and pulsations that resulted from movement of blood in the body.

In other examples, from this heartbeat data, a heart rate can be calculated. A single point sensor can also be used (zero-D), or a linear array can be used (1-D), instead of or in addition to the image sensor (2-D). An image sensor would allow this measurement to be seen at many pixels, allowing a heart rate to be determined across an image.

Further, it is not required that the sensor have contact with the subject. The heart rate sensor could be a white LED mounted in an exercise machine, with an image sensor in the display panel of the exercise machine measuring the exercising subject without contact.

In further examples, the sensor is not limited to measuring the heart rate of a wearer or user. The image could use the same algorithms to extract heart rate from a room full of observers, such as during a poker game or a business meeting, or at an airport checkpoint.

In still further examples, as cardio-workout is defined in terms of minutes of elevate heart rate (either above baseline, or as a percentage of maximum ideal heart rate), one could auto-calculate the minutes of cardio workout in any day, automatically, so that the user does not have to see heart rate graphs or tables, merely seeing just the minutes of ideal cardio-workout per day for example.

Also, from the above examples, it is clear that multiple analyses can be performed on different regions of the sensor, allowing multiple people to have measurements such as heart rate measured for each person either simultaneously, or by selection. The approach is not limited to one target subject, nor to the wearer of the device. The determination could be from a glasses-mounted device that displays the heart rate of those around the wearer, and displays these results for the wearer to view.

In further examples, multiple image sensors could allow such data to be collected from groups of subjects in more than one location, such as from different rooms or different checkout aisles.

In still further examples, note that there is some baseline variation. The size of the pulse signals varies with respiration. Because of this, a respiratory rate signal can be derived, and this can be used to estimate respiratory rate from optical data from wrist, ankle, or face, using measurements obtained even at a distance.

Yet in further examples, such measurements are not limited just to heart rate. Screening for medical diseases (such as anemia, tachycardia, heart rhythm irregularities, jaundice, malaria, heart failure, diabetes, jaundice), chemical levels (alcohol, high cholesterol), or even fitness can be screened.

In other examples, because the measures can be broadband, the background light, which varies according to optical contact or coupling of the light to the subject, can easily be subtracted. For example, a baseline may vary widely as a subject runs and moves with a loose fitting heart rate sensor. However, once the baseline movement is corrected (all wavelengths may change, unlike the heart rate measurement which involves some of the wavelength spectral channels), the background corrected values may more clearly show the hemoglobin variation that represent the changes with heart beats (e.g., heart rate). This allows a non-contact measurement that is resistant to movement, motion, changes in position, changes in background light (such as running in and out of the shadows of trees), all because the broadband values are oversampled, with excess data that allow for background light correction.

In still other examples, because this approach involves broadband light, even background lighting can be used to extract the measures, such as room light in a meeting, or sunlight on athletes working outdoors. This can allow elimination of the white LED.

### EXAMPLE 2

### Content Aware Detection

As an example of content awareness, one use of the detection of these features is the ability to detect tissue.

Conventional proximity detection involves either an intensity measure that changes as tissue moves closer or farther away, or uses a distance monitoring method to detect the distance from the sensor to the nearest object. Both of these approaches have problems. Both of these methods would view a piece of paper moving closer as the same as a face moving closer. That is, they are neither content-aware nor bio-aware.

In a study performed with human volunteers, a hand was moved over a sensor. The presence of hemoglobin at a tissue saturation level expected in human subjects was used as a measure of the presence of living tissue, and the observed intensity of the signal was plotted as a proximity signal. Also calculated was a pure intensity signal, which is the standard proximity signal.

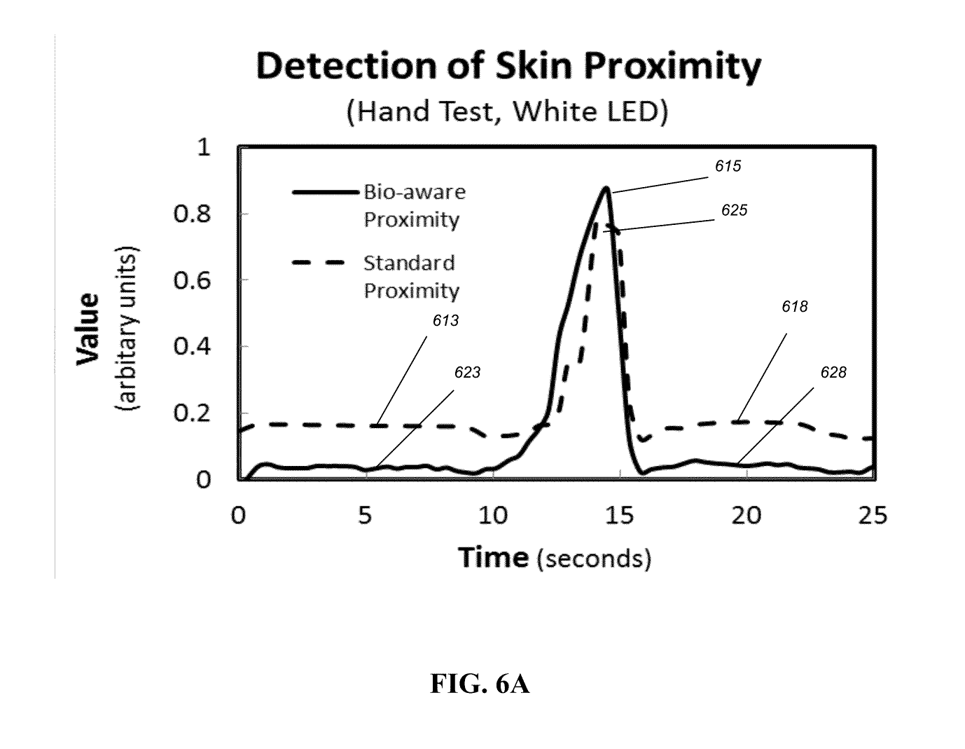

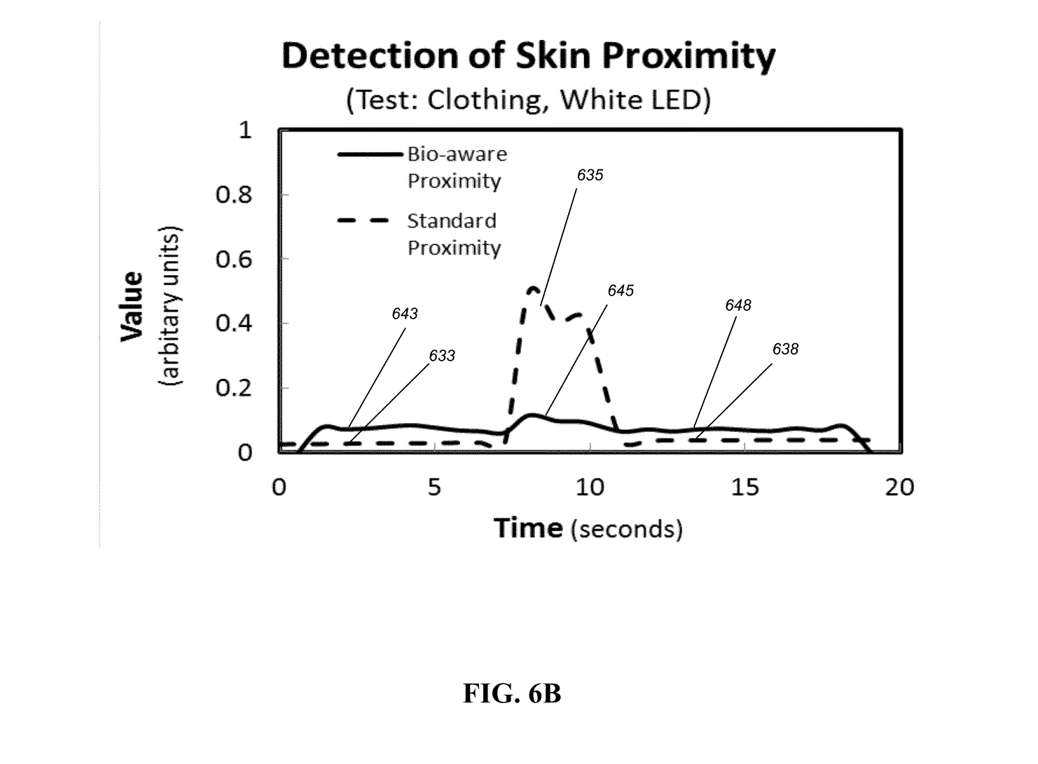

Data are plotted in FIG. 6A-B.

In a first study, data are shown from a hand passing over the sensor, as shown in FIG. 6A. Here, standard proximity signal is shown as a dashed line, starting at a low value before viewing the skin is seen at point 613, then rising to a maximum when the hand is seen at time point 615, then falling again at time point 618 as the hand moves past the sensor. This rise and fall would be consistent with a detection of the hand by a standard proximity sensor. A similar pattern is seen by the bio-aware proximity sensor, starting at point 623, rising to a maximum at 625, and falling again at point 628. In this case, both the standard proximity sensor and the bio-aware proximity sensor return the same result.

In some examples, a study is repeated with a piece of inanimate cloth over the wrist passing over the sensor, as shown in FIG. 6B. Here, standard proximity signal is again shown as a dashed line, starting at a low value before viewing the sleeve is seen at point 633, then rising to a maximum when the sleeve is seen at time point 635, then falling again at time point 638 as the sleeve moves past the sensor. This rise and fall would be consistent with a detection of the sleeve by a standard proximity sensor. In this case, with the skin covered, a different pattern is seen by the bio-aware proximity sensor, starting at point 643, failing to rise to a maximum at 645, and remaining low at point 648. In this case, the standard proximity sensor and the bio-aware proximity sensor return different results, because the bio-aware sensor does not detect any living tissue within the field of view of the sensor. This bio-aware sensing can have many purposes.

For example, a security device could trigger an alarm not just when motion is detected, but when human hemoglobin or a human pulse is detected. This security device could be made to distinguish human hemoglobin from other animal hemoglobin, such that a dog in the security camera view would not trigger an alarm, even if moving. Because the determination can be performed in a non-contact mode at a distance, the technique could be integrated into video cameras, ceiling sensors, lampposts, and the like.

Similarly, the bio-aware sensor could be used to control illumination. In this case, it is not security that is the issue, but energy efficiency. The lights in a room controlled by a motion sensor may turn on when a subject enters, but turn off when the same subject sits still at a computer monitor. A bio-aware device would turn off the lights when the living human leaves a room, and there is no remaining human hemoglobin or human pulse in the room. Similarly, the lights would not turn on when the family dog enters the room, as the detection would be keyed to human physiological features, while non-human hemoglobin is often spectrally quite distinct from human hemoglobin.

Next, the device could distinguish between real and sham tissue, such as for unlocking security sensors that are image based (such as fingerprint sensors that can be fooled by photocopies of fingerprints).

Next, the device could be used to turn on or off phones when the screen is placed against a face by detection of the human tissue.

Next the sensor could be used to detect where a laptop or tablet is being held, to distinguish human touch from the pressure of a pocket or table.

Last, because different people have differing body composition (fat/water/melanin), different skin thicknesses, different levels of tanning, are of different races, age, gender, and ethnicity, this content awareness could provide some identification features. For example, even without a fingerprint being entered (for instance, if a cell phone is unlocked but is grabbed or picked up by an unauthorized user), then the normal composition of the user in terms of the above characteristics could be used to identify the user, and lock out an unauthorized user who is holding the phone. Similarly, markers (such as dyes, tattoos, unique mixtures of quantum dots) and the like could be used to make very specific optical markers that are nearly impossible to forge, due to the large number of admixtures of different wavelengths of quantum dots (perhaps hundreds could be distinguished) as well as each type having a relative ratiometric concentration, sensitive to one part in 2 raised to the 16^{th} power, or more. As each agent could be in various concentrations, this alone would yield 2 to the 20^{th} mixtures, even without a spatial tattoo patterning. Such implanted dyes could be encapsulated to be stable, providing non-radiowave, optical identification difficult to reproduce or transfer. Combined with a live dead detection, a high level of security could be achieved.

### EXAMPLE 3

### Heart Performance from a Bracelet Monitoring

In some examples, a bracelet was constructed using a white LED light and an optical fiber. The optical fiber allowed for ease of construction, in that a silicon sensor did not need to be incorporated into the small wristband. Rather, the light was transferred from the optical fiber to a commercial spectrally resolved linear sensor and measurement system (T-Stat 303) operating in a data-recording mode.

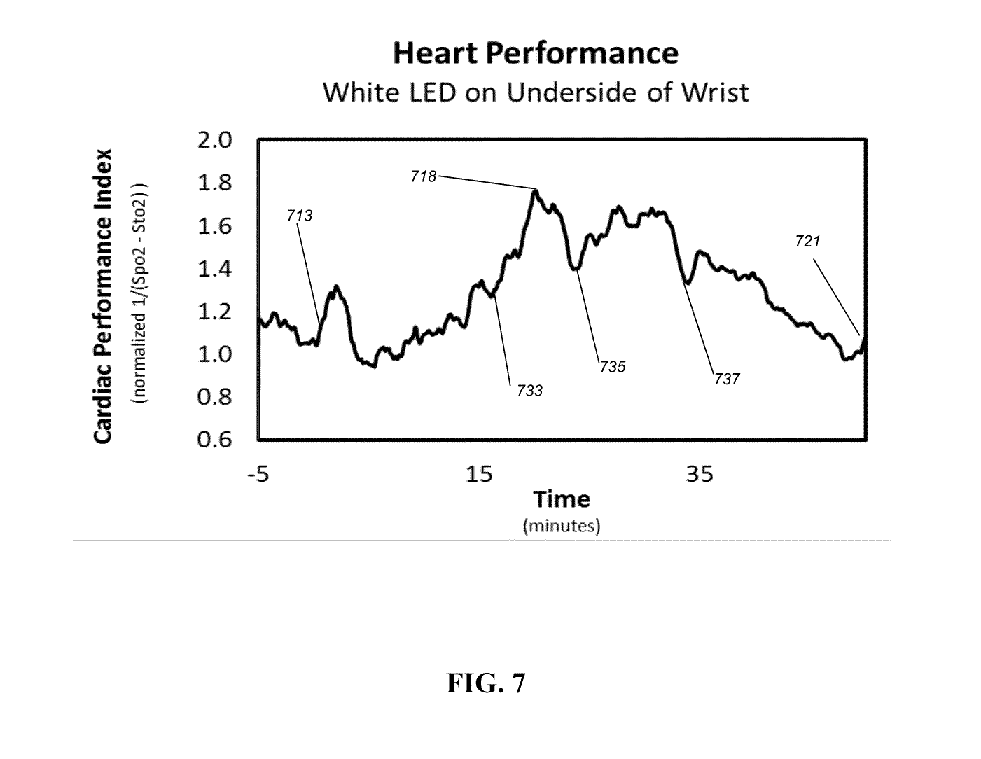

In other examples, a fit subject may be exercised on an elliptical trainer. The power of the workout (joules/hour), the subject's heart rate, respiratory rate, work power, and pulse oximeter reading were recorded using other monitors, including a video recording for synchronization of the various data during analysis. Selected resulting data are plotted in FIG. 7.

In FIG. 7, a measure of cardiac performance is calculated, as the reciprocal of the arterio-venous difference, defined as [1/(SaO2%−SvO2%)]. For this, the SaO2 % was estimated using a pulse oximeter, SvO2% was estimated from tissue oximetry of the wrist from data collected from the bracelet using known SvO2% determination algorithms from spectral data, and the data were normalized to 1.00 at the start of the study. The SvO2% measurement was performed using spectral fitting to data from the wristband using tissue oximetry. In practice, a wristband would use the same approach as shown in Example 1, using a white LED, a silicon imager and a spectral filter, and least squares fit of the spectral data using oxygenated and deoxygenated hemoglobin standards.

Data are shown in FIG. 7. Here, cardiac performance is 1.00 at the start of the study, at point 713. As the subject begins to exercise, performance rises to peak at point 718, then returns to near baseline after recovery to point 721. Also seen are 60-second rest periods at time points 733, 735, and 737. Even during the short rest period, the recovery of heart function is seen. Note also that during this exercise recording, a pulse oximeter readout (medically called SpO2%) remained at 96-98%, and also that the heart rate measured did not recover substantially at all during the rest periods (not shown). There were large dropouts in which the pulse oximeter further was unable to read at all due to motion artifact.

Last, taking the power of the exercise in joules/hour (as measured from the elliptical trainer, which is an estimated workload in this case as this trainer was a commercial exercise device not a physiology lab device, though we expect the power estimates to roughly track a physiology device) and correlating with the cardiac performance on a scatter plot shows that among heart rate, pulse oximeter, and cardiac performance measures, cardiac performance correlates well with workload (r²>0.82).

There are several points to note here

First, this data was collected with a fiber-based system for ease of laboratory analysis. Use of a mobile system with an LED and a sensor would be one approach to measure these values on mobile athletes. The use of a tethered fiber-optic wrist probe was for proof of feasibility.

Next, cardiac performance could be one of the first performance based devices available to athletes that measures cardiac performance using a simple, optical, non-contact, wrist-based monitor.

The form of a monitoring device includes non-contract pendants, cameras, phones, wristbands, and other wearables. The sensors could be incorporated into clothing such as gloves, spandex suits, caps, bracelets, pendants, and the like.

### EXAMPLE 4

### Body Composition on a Dieter

Hemoglobin is one of the most intense and visible pigments in the body, however there are many other pigments that can be measured by this method.

Fats and water are key body constituents, and have spectral features. Fats exhibit a peak at 920 nm (and elsewhere, including near 760 nm), while water has a peak at 960 nm (and elsewhere, include second differential peaks about 820 nm, large absorbance peaks between 1 and 2 microns, and a broad absorbance peak more or less between 2 and 10 microns).

We constructed a device that measures in the infrared by modifying a commercial spectral monitor (T-Stat 303) to measure on the body. This device has a broadband infrared LED instead of a broadband white LED.

The table below shows determinations from this system, which measuring on a hand, wrist, breast, and head, as shown in Table 1, below:

TABLE 1
Components of living tissue include fat and water. Other substances,
such as volume of bone, collagen, and pigments such as melanin and
heme, therefore the values do not sum to 100%. Multiple measures
around the body could allow for body composition analysis.

Material

| Tissue | Fat | Water |
|---|---|---|
| Finger | 12% | 65% |
| Breast | 45% | 22% |
| Bicep | 15% | 61% |
| Abdomen | 33% | 42% |
| Ankle | 18% | 55% |

### EXAMPLE 5

### Discrimination of an Organic Finger vs. a Non-Living or Inorganic Sham Finger

Security systems require an identifier in order to detect the presence or identity of a person. Sometimes this identifier is a password or ID chip, while at other times it is a biometric measure (fingerprint, retinal blood vessel pattern). However, some fingerprint detectors can be fooled by something as simple as a cyanoacrylate copy of a fingerprint on cellophane tape.

By performing the analyses of the above examples (detection of heart rate, cardiac performance, fat/water composition), one can easily distinguish real from sham tissue.

In this example, we perform the measures listed in the above example. Tissue is measured for hemoglobin (heme) content. Normal tissue is 20-120 uM heme, with a saturation between 30%-80% for SvO2%. Further, living tissue is mostly water and fat, with water and fat comprising 50-90% of the volume in sum total. Further, there should be a low fitting error (for this algorithm, the error from unrecognized components should be below 200 though this number may vary by system and algorithm). Once these features are taken into account, the real, live tissue (as opposed to dead meat, colored paper, or inanimate objects) can easily be recognized, as shown in Table 2, below:

TABLE 2
Once the components and features of living tissue are taken into
account, the real, live tissue (as opposed to dead meat, colored
paper, or inanimate objects) can easily be recognized.

| Tissue/ | | | | | | | | |
|---|---|---|---|---|---|---|---|---|
| Mate- | | | | Has | | Wa- | Fit | Live |
| rial | Heme | | Svo2% | Pulse? | Fat | ter | Error | Tissue? |
| Finger | 51 | uM | 55% | Yes | 12% | 65% | 68 | Yes |
| Breast | 20 | uM | 71% | Yes | 34% | 22% | 91 | Yes |
| Meat | 450 | uM | 0% | No | 15% | 61% | 122 | No |
| Table | 2 | uM | n/a | No | 0% | 0% | 45341 | No |
| top | | | | | | | | |
| Red | 1 | uM | n/a | No | 0% | 0% | 3911 | No |
| Paper | | | | | | | | |

The “n/a” value indicates no value is determined when the material is not human tissue with blood.

Different subsets of this approach can be taken into account, depending on application. For example, a pulse (heart rate) takes a few seconds to detect, while fat and water can be measured in a microseconds. Therefore, a fingerprint sensor that seeks to verify what is alive and not alive, or real and not real, may wish to use the spectrally determined composition in this analysis.

### EXAMPLE 6

### Incorporation Into Systems and Devices

The sensor as described can be incorporated into a small sensor or device.

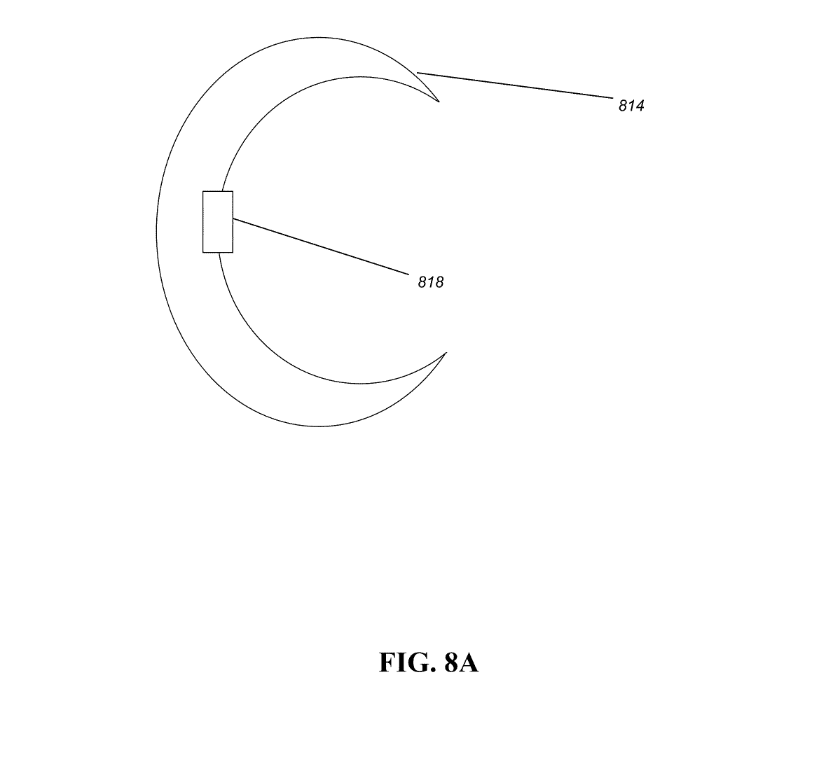

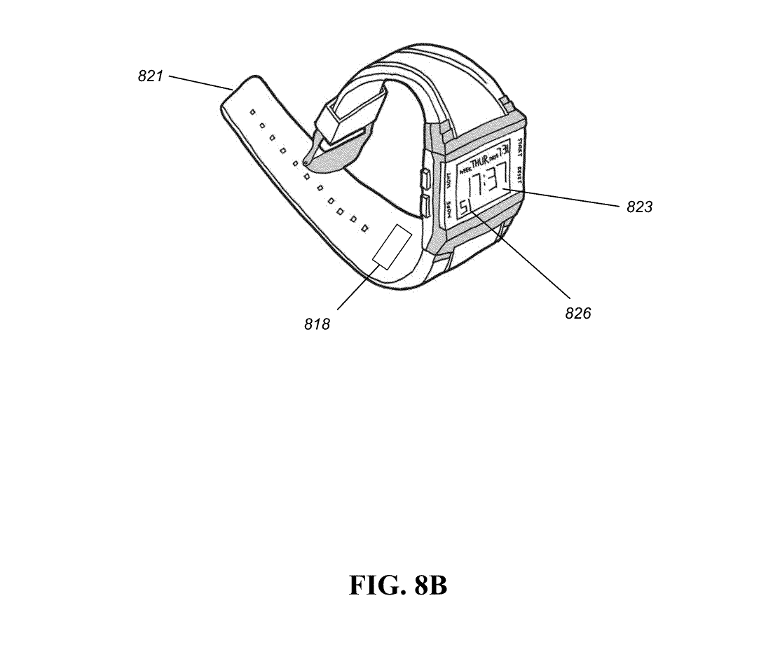

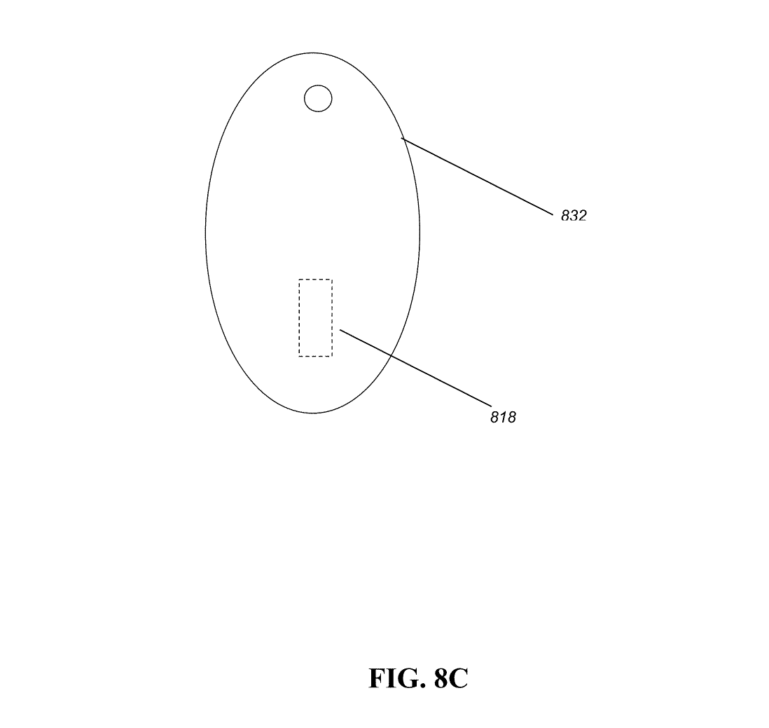

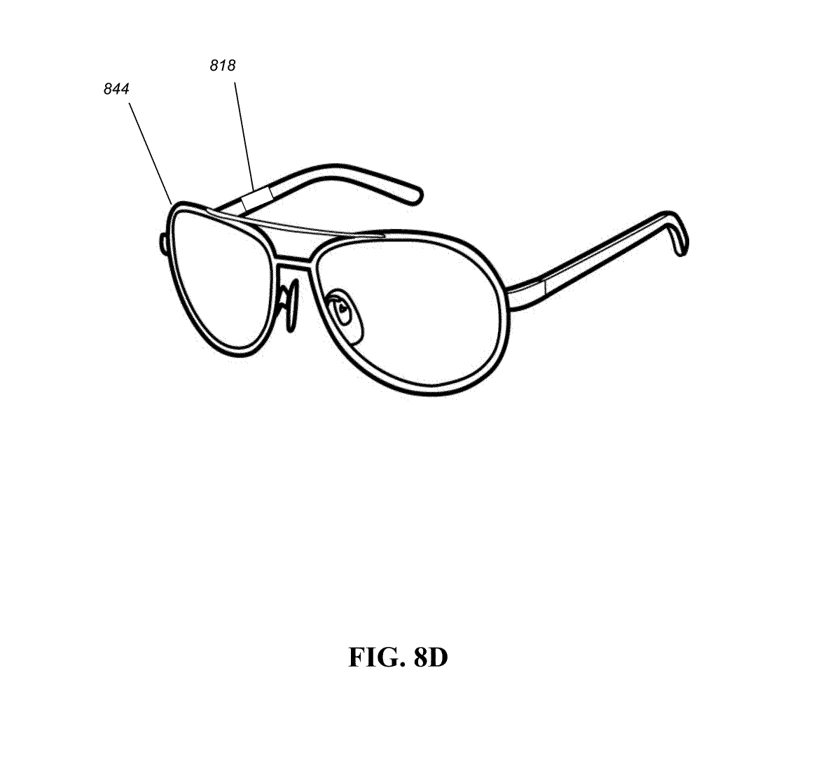

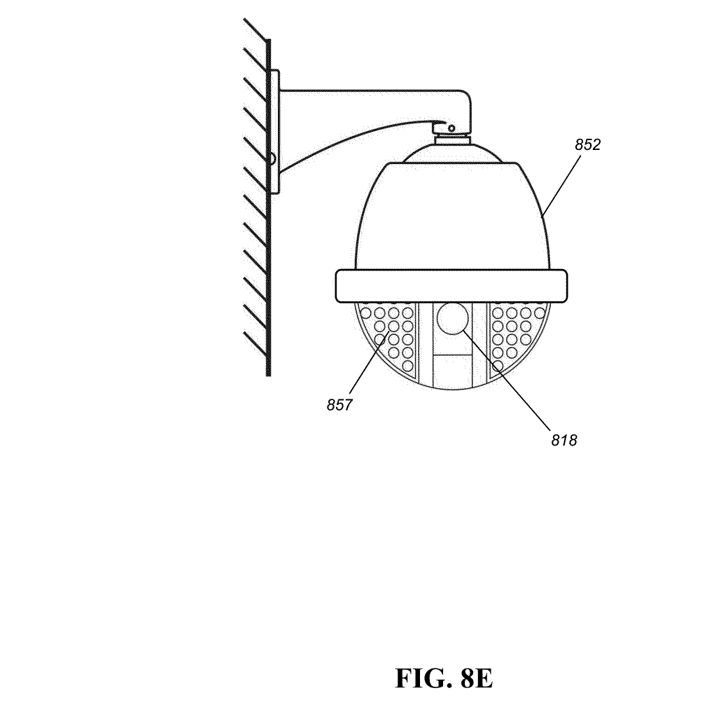

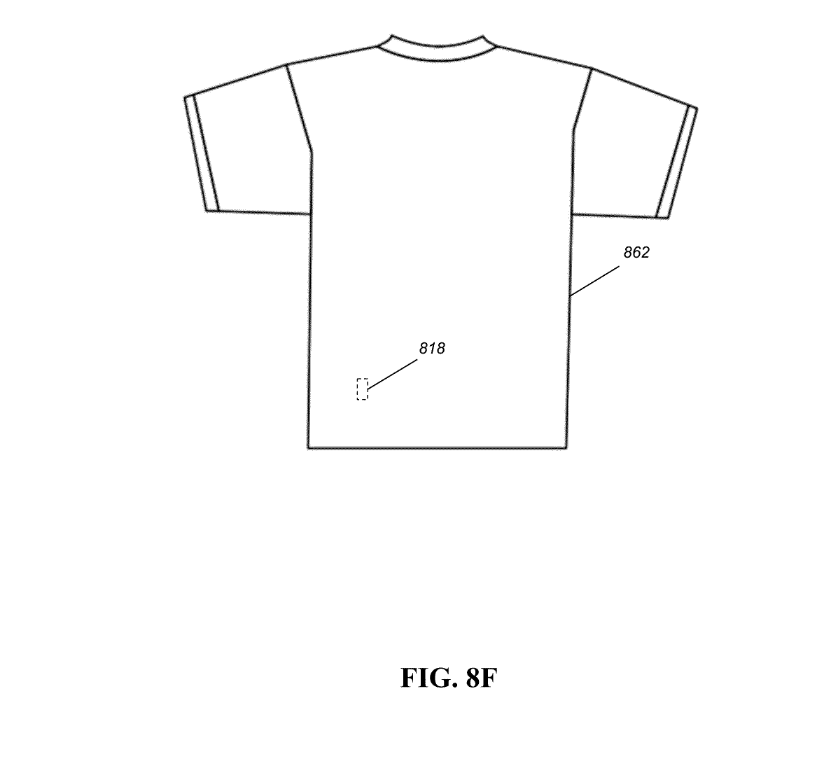

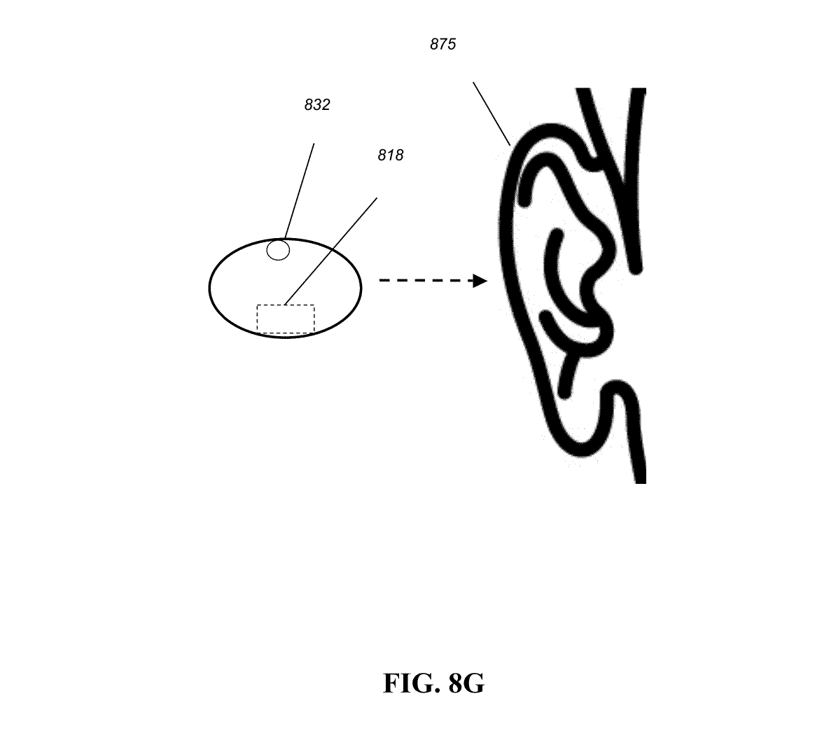

Several devices incorporated into systems are shown in FIG. 8A through FIG. 8G.

A loose fit wristband is shown in FIG. 8A. Here, loose-fit wristband 814 has sensor 818 integrated into its body. This would allow a fitness band, as well as a monitor for persons with chronic medical disease.

A medical or fitness wristwatch is shown in FIG. 8B. Here, wearable watch 821 has sensor 818 integrated into its body or strap. Display 823 shows a user certain useful information, including heart rate 826. This would also allow for a fitness band, as well as a monitor for persons with chronic medical disease.

A heart-rate sensing pendant is shown in FIG. 8C. Here, pendant 832 could hang near the users' body, but not in fixed or permanent contact with the skin, and has sensor 818 integrated into its body. Such sensors could be on two sides, such that one side always senses skin. The proximity sensing and tissue sensing disclosed within could turn on the side against tissue.

Wearable glasses with sensor are shown in FIG. 8D. Here, wearable glasses 844 have sensor 818 integrated into frame or lenses. A display could be added, much as in heads-up displays to show a user useful information, including heart rate, or into a device such as Glass (Google, Mountain View, Calif.). The sensor can look outward as well, and record heart rates in business meetings, road races, and the like. As noted earlier, the face is a strong source of heartbeat pulses, and the decreased motion compared to the legs and arms makes this an excellent source of measurement.

A remote sensor for ceiling or rooftop mounting is shown in FIG. 8E. Here, remote sensor 852 has sensor 818 integrated into its body or strap. Additional white LED or infrared illumination is provided by LED array 857.

A wearable clothing sensor is shown in FIG. 8F. Here, shirt or textile 862 has sensor 818 integrated into the textile. Wireless communications could be added to communicate with other devices, such as watch 821 or glasses 844 of FIG. 8G, or cell phone 101 of FIG. 1.

An insertable ear probe, into which a heart rate sensor could be placed, is shown in FIG. 8G. Here, earbud 875 has sensor 818 integrated into its body or strap. As noted earlier, the ear is a strong measurement source, though this varies from the pinnae to the auricles to the external canal.

One point of note, different parts of the body have stronger or weaker signals, depending upon what is being sought. For example, the pulsatility at the wrist is often lower than at the fingertips, nail beds, ear lobes, lips, cheek, or forehead, while the ability to measure subcutaneous fat is better over the wrist than in the lips. In contrast, the face has a different venous pulsation with movement than does the wrist. In part, this has to do with the blood flow of the tissue, and the thickness of the skin, but it also is affected by the venous valves present in the arms, but not in the face. Because of this, different sensor configurations, and different algorithms, may be used at different places.

### EXAMPLE 7

### Non-Contact Sensor Design

In this description the terms loose-fit and non-contact are used. Light forced into tissue (such as from an emitter in physical contact with optical elements of the emitter directly into tissue) and detected by an emitter also in direct physical contact with tissue (such as a CCD pressed directly against skin) travels a different average path than light coming from an emitter source, travelling through the air to skin or tissue, and then scattering and reflecting back to an emitter, also at a distance from the tissue. Further, direct pressure to the measured tissue can suppress pulsatility (though minor pressure may suppress the effects of movement more than the pulsatility).

One way to encourage or ensure the system is non-contact is to place the sensor into a device intended to be kept at a distance, such as cell phone 101 of FIG. 1, or ceiling security sensor 852 of FIG. 8E.

However, such distance is not always possible, especially with wearable devices. In such cases, it may be important at times to force the sensor to remain out of physical contact with the subject, tissue, or object to be examined. In such cases, a design as shown in FIG. 8A through FIG. 8C, or the ear buds of FIG. 8G, may be advantageous.

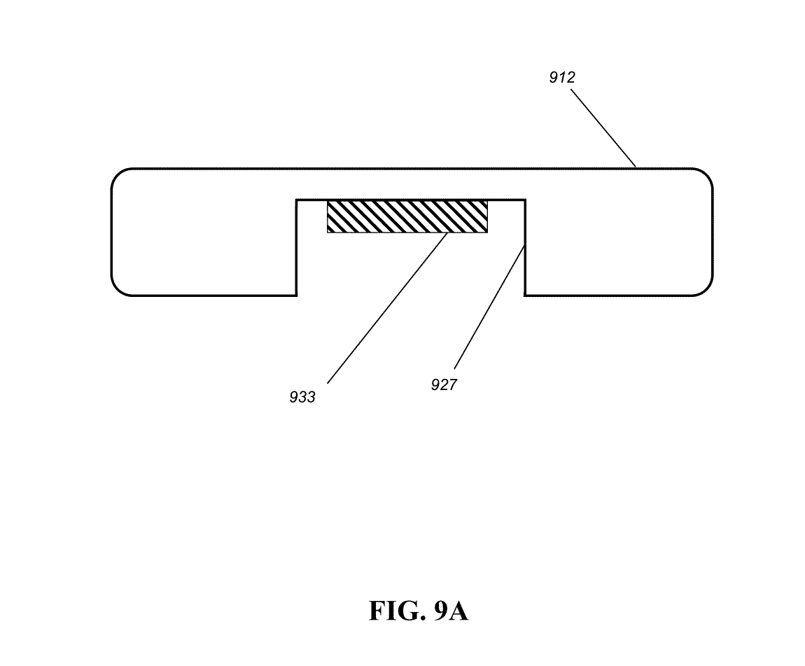

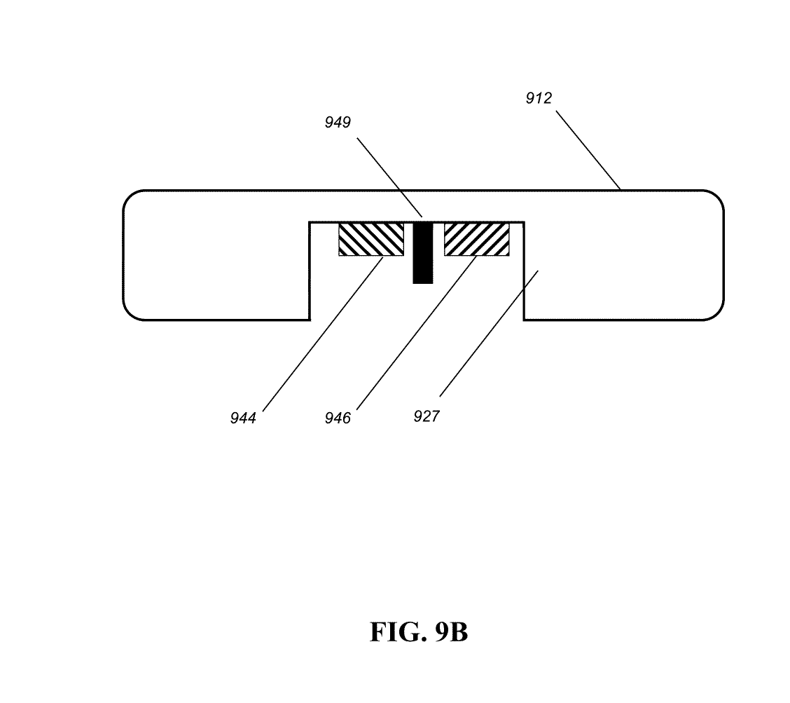

Such a hardware method to ensure the sensor is non-contact is shown in FIG. 9A and FIG. 9B.

First, a recessed non-contract sensor with the illumination and detection on the same chip are shown in FIG. 9A. Here, device 912 has well 927 which holds sensor 933. Well 927 holds sensor 933 away from the skin, by millimeters to centimeters, making light reflect off of the tissue or objects surface when device 912 is held against the tissue or object.

Alternatively, sensor 933 can be separated into separate components, such as emitter 944 and detector 946, with light shield 949 between the two, as shown in FIG. 9B.

Note that in these designs, emitter 944 and/or detector 946 may also each be composed of multiple components that are also similarly separated.

### EXAMPLE 9

### Measurement of Respiratory Rate

Breathing leads to increases in pulse size at a time constant determined by the breathing rate, as well as shifts in venous blood proportionate to the depth and effort of respiration.

During inspiration (breathing in), the pressure in the chest cavity drops, increasing the rate of return of venous blood to the heart. This in turn makes the pulse volume larger, as cardiac output volume for each beat is driven in part by how much blood returns to the heart during filling during the rest cycle. As a result, the pulse size rises and falls with respiration. This produces a volume change in the total arterial blood signal that has frequency of 8 to 30 times a minute (even faster in infants). By analyzing the average beat-to-beat volume changes in the arterial compartment at longer frequencies than typically seen for heartbeats, a respiration measure can be seen and counted. Averaging for 0.5 to 2 seconds (or frequency filtering) smooths out the pulse, and allows changes in the arterial pulse size to be determined.

Arterial compartment data from exercising human subjects as determined in the previous examples were analyzed using increasing smoothing on the arterial signal, which focuses on the respiratory changes. The respiratory changes can be considered another physiological compartmental contribution (that is, a first compartment with the heartbeat, having a fundamental rate of the heart rate, and a second compartment with the respiratory effect, having with a fundamental rate of the breathing cycle).

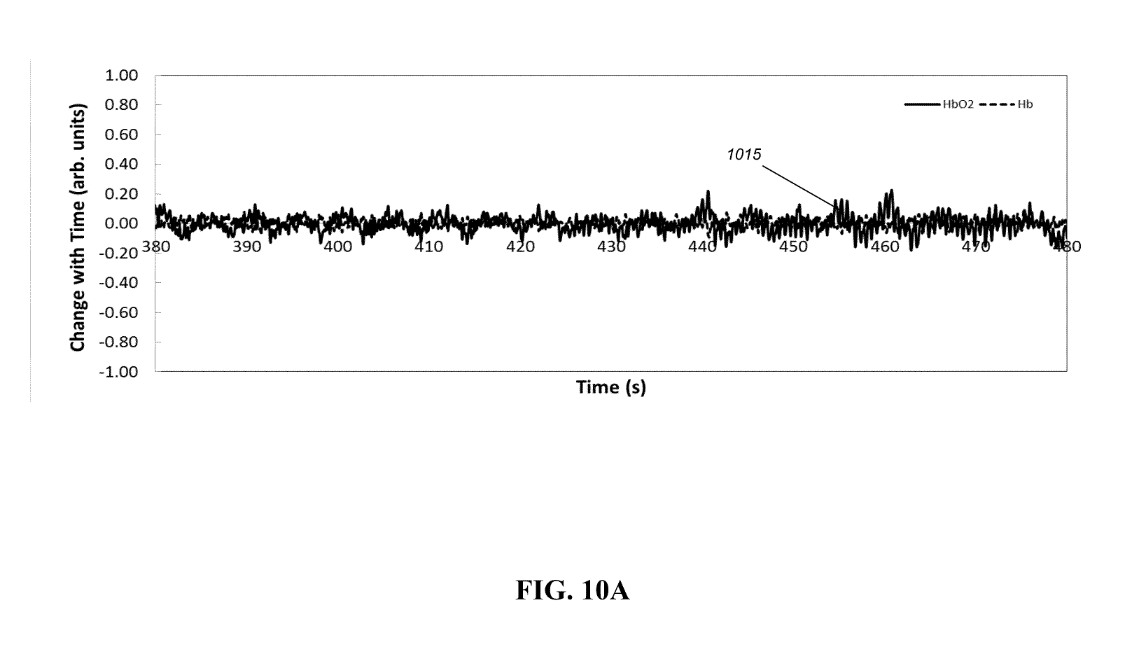

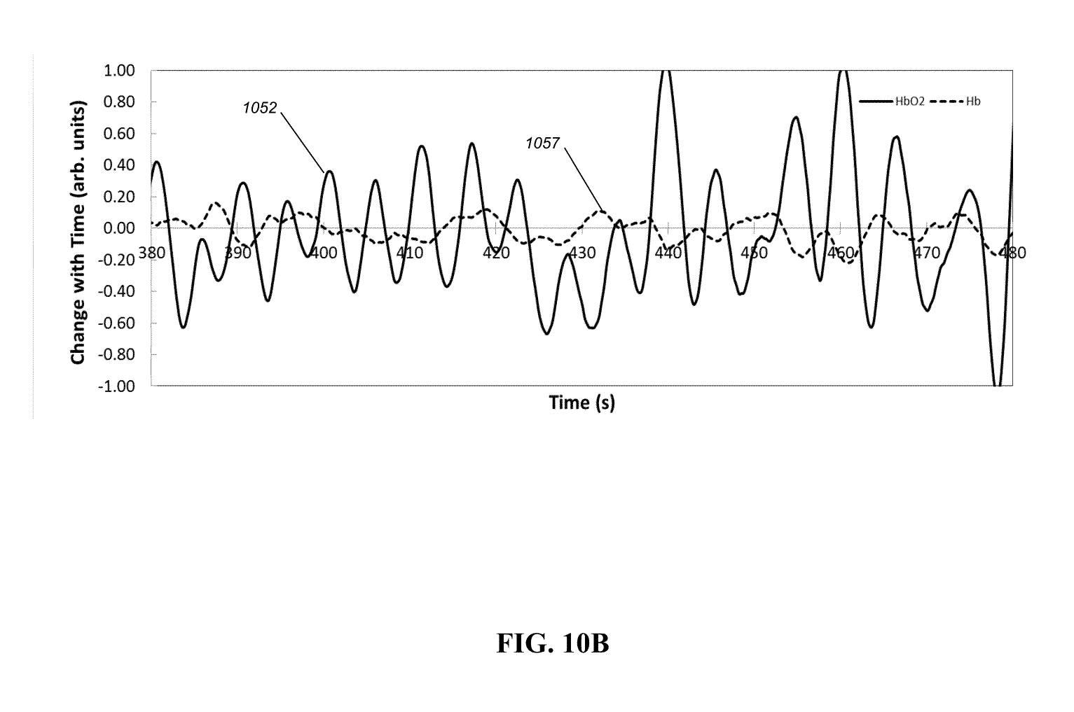

Data are shown in FIG. 10A-B. Here, oxyhemoglobin and deoxyhemoglobin changes over time were initially calculated as in the previous examples. However, different time constants are applied in FIG. 10A and FIG. 10B.

In FIG. 10A, arterial pulse data are shown during exercise (jogging from 380 to 420 seconds into the study) and through the transition to standing still (still from 420 to 480 seconds) in graph 1010. There is little baseline change in the blood because of the previous multi-spectral processing. There are many fine spikes, such as the spikes seen at time point 1015, which represent the heart rate in the arterial signal. These heart rate effects are difficult to see due to the scale, but note that the oxygenated and deoxygenated heme signals are both shown. The time constant for this data is change over a 150 milliseconds with 30 millisecond data sampling.

In FIG. 10B, the same data from FIG. 10A are shown, but subjected to a different time filtering. Here, the data are high-pass filtered with a time constant of 2 seconds, shown in FIG. 10B as graph 1050. Now, the respiratory effect dominates the oxyhemoglobin curve 1052 (solid-line), but is minimally present in the deoxygenated hemoglobin curve 1057 (dashed-line). Counting these cycles shows a respiratory rate of 18 breaths in 100 seconds, or about 14/minute. Further analysis (not shown) into compartments shows the respiratory effect is seen to be isolated to the arterial compartment.

Several points are worth noting in discussion.

First, these signals can be increased when breathing hard, and therefore the size of the signal increases during hard exercise. The signal is also increased during certain respiratory diseases, such as congestive heart failure (due to pulmonary edema), asthma (due to obstructive pulmonary disease), and choking (due to increased respiratory effort and pressure gradients). One should be able to detect and count coughing, sighs, sneezes, hiccups, and other respiratory anomalies.

Second, by adding another time-constant compartment to the data analysis, the typically 8-30 Hz respiratory signal can be isolated. Similarly, this can be done through Fourier Transform time filtering as well, as is known in the art of time-analysis.

Third, intervals can be used to derive rate, as described below. For example, an estimated heart rate (in beats per minute) may be determined as 60/interval, where the interval is expressed in seconds.

### EXAMPLE 9

### Algorithm

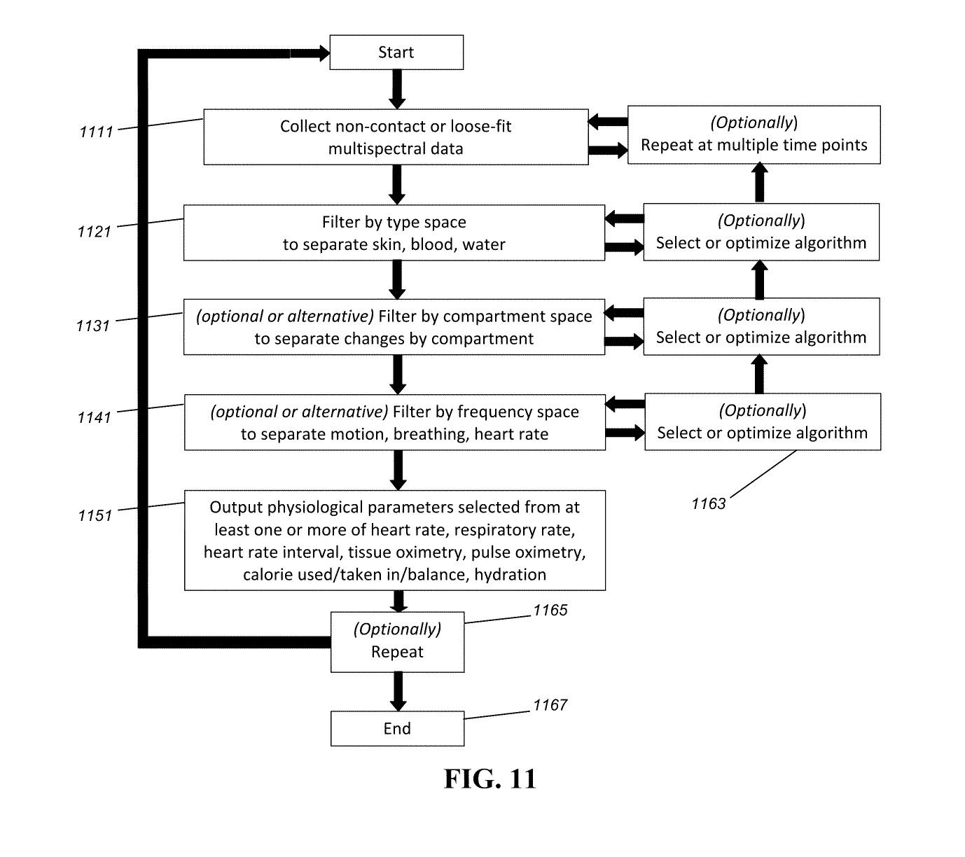

An exemplary algorithm is described in connection with FIG. 11. In some examples, there are many ways of performing the described algorithm (i.e., method or process), but, in some examples, provided a multi-spectral and/or multi-compartmental method is used to separate the signals in order to produce a stable method insensitive to motion and/or changes in body position, whether in contact or in non-contact modes, these may be included within the described techniques.

A first step is collection of the data, shown as method step 1111. In the described techniques, the data may be either non-contact optical data or loose-fit data, with a key feature being that multiple wavelengths are used. For complex determinations, this could be 6 or more wavelengths, but for the purposes of this exemplary technique 3 or more is more typical. Subsequently, data may be filtered. In some examples, one or more filters may be used.

One such filter is to separate multispectral data into types of tissue, shown as method step 1121. This may be performed using a matrix fit to the coefficients for the various components using published spectral weights, as was shown earlier. Alternatively, partial least squares (PLS), principal component analysis (PCA), or iterative methods could be used in such solutions.

Another such filter is to partitioning the concentrations or features found by multispectral fitting into different compartments, such as partitioning oxyhemoglobin, deoxyhemoglobin, water, or other substances into arterial and venous compartments, shown as method step 1131. In one example, shown earlier, using values of 70% saturation of the venous blood, and 98% saturation of the arterial blood, the oxy- and deoxy-hemoglobin changes can be seen to occur in arterial and venous compartments.

But there are other phases that can be exploited. For example, there are also venous changes that occur during heartbeats and respirations, with slightly different time constants and phase offsets than the arterial pulse. Also, just as breathing in lowers the intra-thoracic chest pressure, which increases the filling of the heart and produces larger arterial pulses, there can be venous changes as a result of the rising and falling back pressure occurring at the frequency of respiration. Next, body motion, such as raising or lowering an arm, changing body position, or jumping, produces a change in venous blood volume in the tissue (and a smaller arterial change, as arterial blood is higher pressure in muscular arteries, while venous blood is low pressure in floppy vessels). You can see this change by eye when you lower your hand, and your veins become fuller in the back of your hand, while when you raise your hand the vessels collapse and such slow changes are also seen in the studies presented earlier. Because these occur over time, and not instantaneously, there are phases and time constants that can allow identification of additional compartments. Similarly, while the changes that occur with changes in position, or with movement, or with jumping, are largely venous changes, there are some lesser arterial changes, and more sophisticated compartment models may identify these, provided sufficient wavelengths are used.

In each of these cases (heartbeat, respiration, body position changes, movement, and impact from exercise), treating the tissue as having one or more arterial changing component and one or more venous changing components allows for a method of extracting and solving for each of these changes. Each of these compartments is another “unknown” to solve for, and solved by adding more wavelengths. Another unknown, baseline reflection signal, can be solved for using more wavelengths.

Another such filter is to filter in frequency space, such as to separate heartbeat from respirations (effectively two compartments), or even to separate motion (such as probe motion) effects based on their own rhythmic frequencies, as shown in method step 1151. This was shown earlier for separation of heartbeat and respirations using different time constants, but there are many methods such as Fourier Transform or its equivalents to produce a frequency-space data set. Suppression or removal of certain frequency ranges, and back conversion to spectral data would effectively separate the heartbeat and respiratory compartments, and may also be used to remove rhythmic exercise effects, such as walking or running induced probe and body motion.

Last, the entire process may be repeated, as shown in method step 1165, or one or more of each of the method steps can be repeated or used to feed back into prior analyses in order to iteratively improve the results, as shown in method step 1163. At some point, the method is ended, at method step 1167. The ending could be a firm end to calculation, or it could be restarted as needed.

First, other ways of processing can be envisioned, for example an iterative or more sophisticated model may consider the influence of each compartment on the measurement of the other (such as if the arterial component is NOT 100% oxyhemoglobin).

Second, there are other substances that can be measured. Water, for example, can be measured using water peaks (such as at 960 nm or 820 nm) or any other point provided there is measureable contribution in the absorbance signal from water. Similarly, Ethanol, cholesterol, blood lipids, carotene, even medications can be measured in this manner.

Next, heart rate can be collected as an image, allowing the heart rate to be extracted from multiple persons in an image. Thus, a single point sensor can also be used (0-D), or a linear array can be used (1-D), instead of or in addition to the image sensor (2-D).

Next, it is not required that the sensor have contact with the subject. The heart rate sensor could be a white LED mounted in an exercise machine, with an image sensor in the display panel of the exercise machine measuring the exercising subject without contact.

Next, the sensor is not limited to measuring the heart rate of a wearer or user. The image could use the same algorithms to extract heart rate from a room full of observers, such as during a poker game or a business meeting, or at an airport checkpoint.

Also, as cardio-workout is defined in terms of minutes of elevate heart rate (either above baseline, or as a percentage of maximum ideal heart rate), one could auto-calculate the minutes of cardio workout in any day, automatically, so that the user does not have to see heart rate graphs or tables, merely seeing just the minutes of ideal cardio-workout per day for example.

Also, from the above example, it is clear that multiple analyses can be performed on different regions of the sensor, allowing multiple people to have measurements such as heart rate measured for each person either simultaneously, or by selection. The approach is not limited to one target subject, nor just to the wearer of the device. The determination could be from a glasses-mounted device that displays the heart rate of those around the wearer, and displays these results for the wearer to view.

Next, image sensors could allow such data to be collected from groups of subjects in more than one location, using the pixels for each subject studied to calculate that subjects physiology data, such as from large rooms, street corners, security lines, or checkout aisles in stores.

Next, such measurements are not limited just to heart rate. Screening for medical diseases (such as anemia, tachycardia, heart rhythm irregularities, jaundice, malaria, heart failure, diabetes, jaundice), chemical levels (alcohol, high cholesterol), or even fitness can be screened.

Next, because the measures can be broadband, the background light, which varies according to optical contact or coupling of the light to the subject, can easily be subtracted. For example, a baseline may vary widely as a subject runs and moves with a loose fitting heart rate sensor. However, once the baseline movement is corrected (all wavelengths may change, unlike the heart rate signal which involves some of the wavelength spectral channels), the background corrected signal may more clearly show the hemoglobin varying signal of the heart rate. This allows a non-contact measurement that is resistant to movement, motion, changes in position, changes in background light (such as running in and out of the shadows of trees), all because the broadband signal is oversampled, with excess data that allows for background light correction.

Last, because this approach involves broadband light, even background lighting can be used to extract the measures, such as room light in a meeting, or sunlight on athletes working outdoors. This can allow elimination of the white LED.

### EXAMPLE 10

### Separation into Compartments

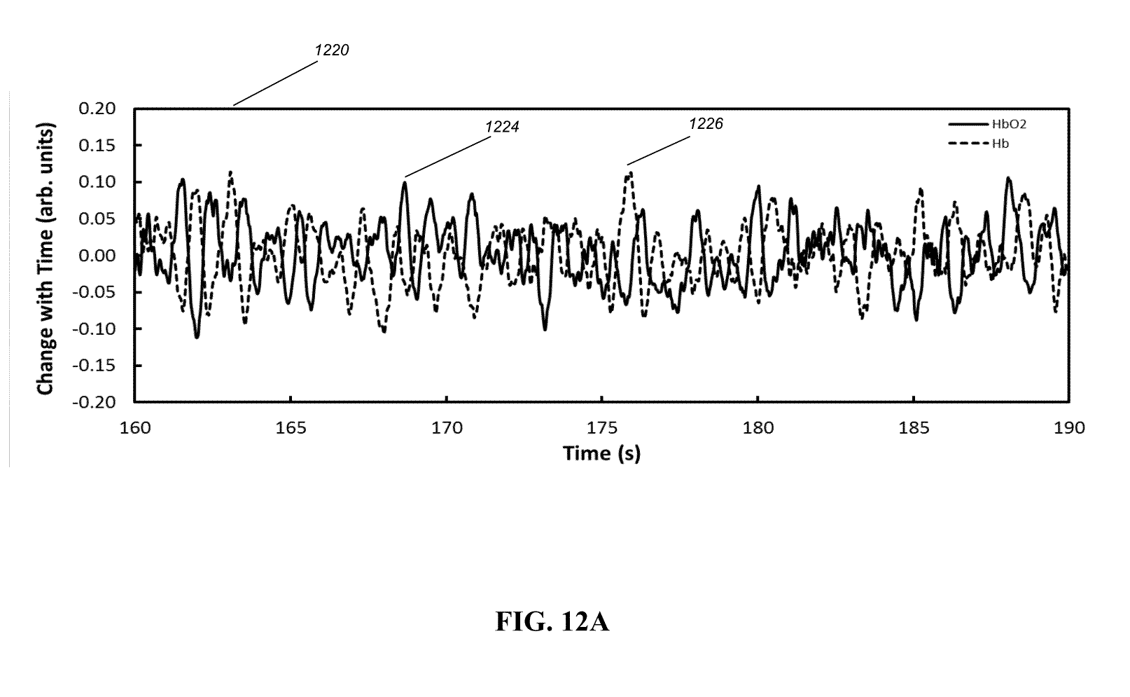

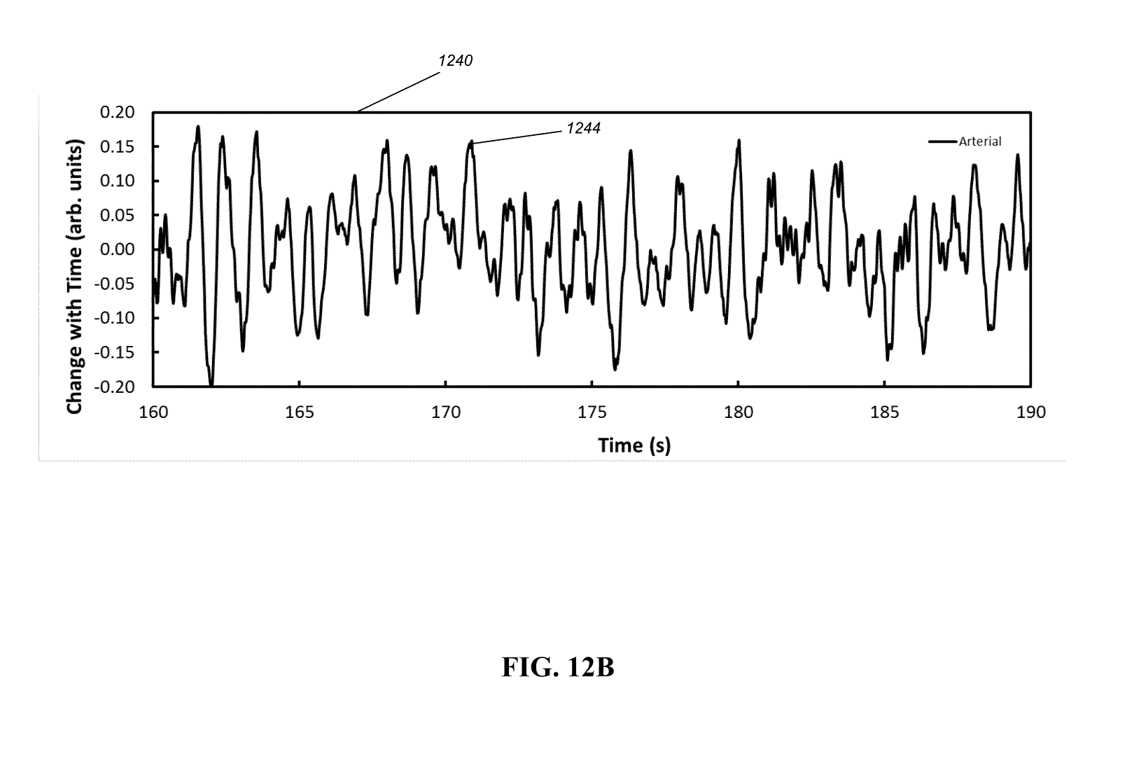

This approach can be applied to human data collected under study conditions. Multi-spectral analysis of that spectral data, in this case through a matrix solution of simultaneous linear equations, yields the data shown in FIG. 12A-B. Here, plot 1220 of FIG. 12A shows hemoglobin concentration changes over time at the transition from stillness to exercise at 180 seconds, analyzed and re-plotted for 160 to 190 seconds with tissue contact changes and non-heme components minimized by differential analysis, plotted for changes in hemoglobin concentration over time. The oxyhemoglobin concentration (shown as solid line 1224) and the deoxyhemoglobin concentration (shown as dashed line 1226) can be seen to vary differently. These two plots differ in degree of change, timing of peak changes, and even frequency, which clearly demonstrates separation of different signals that change at different times.

Now, data is further analyzed by blood compartment. As described earlier, the venous compartment which is affected more by gravity, body position, and impact, while the arterial compartment which is affected more by heart rate and respirations. Separation of these compartments with further analysis is shown as plot 1240 of FIG. 12B. Here, arterial-only plot 1244 is shown.

One key to the compartment separation is that arterial and venous blood have different oxygenation. In this example, we assume that the arterial compartment has a heme saturation of nearly 100%, while the second, venous compartment has an oxygen saturation of 70%. This separation yields an arterial-only volume curve shown as graph 1240 in FIG. 12B. In this graph, the artifacts and noise from body movement and probe movement are nearly gone from the arterial pulse signal. Thus, solving for different compartments therefore allows a pulsatile arterial component, with a heartbeat associated more or less with each of the arterial local maximum values, to be separated from a widely varying venous component. Note that a large change in blood volume and absorbance is weakly seen visible in FIG. 12A and FIG. 12B, and further that the pulse peaks are clearly seen even at 180 seconds and after, well into movement and/or exercise, in FIG. 12B.

In the calculations of this example, a simplistic but fast way to solve for the compartments was to consider venous blood to be 70% saturated, and for arterial blood to be exactly 100% saturated. Solving for deoxygenated blood yields changes that may be venous, as arterial blood has no venous blood in this simplistic analysis. Since venous blood is 30% oxygenated and 70% deoxygenated, the amount of total amount of venous blood changes can be calculated from the deoxyhemoglobin change plus an additional volume change of 30/70th of the deoxyhemoglobin change (that is an additional 30% volume that is oxygenated for every volume of venous blood that is deoxygenated). Removing the oxygenated component of the venous blood leaves a change in this example that may be the arterial compartment change, which is far more pulse-driven than gravity- and body-position-driven. This allows a pulse to easily be seen, as shown in FIG. 12B.

First, it is important to note that such a 70%/100% assumption is not required, and even iterative methods can determine the ratios that best fit the data.

Second, mathematical methods of solving such multiple equations are known. For example, one can apply multiple linear equations, where the values in the equation are: (1) an array of measured data within each waveband, (2) the corresponding absorbance, such as blood with and without oxygen, bilirubin, water, or fat, and (3) the result vector, which yields the concentrations (or changes in concentration) over time. In such an example, if the measured data is an N-element 1-D array named B, representing the data measured at N wavebands, and the known coefficients of effective reflection absorbance (absorbance and scattering) of each of M substances at each of the N wavebands are in a M by N 2-D array (a matrix of coefficients) named A, while the concentrations of each substance to be determined are in an M-element 1-D array of unknowns called X, then the values of X can be determined as (after regularization such that the math works, such as making N=M) then X equals the matrix operation: A⁻¹B. The values for the array of coefficients can be found in publications, or may be experimentally estimated. Alternatively, simple algebra can be used to reduce the complexity of the calculations to mere ratios in certain conditions, or weighted nodal partial-least-squares analysis can be used for even a more complex analysis. All of these fall under the present exemplary technique if used to correct for distance and motion in a loose-fit or non-contact physiological monitoring.

As another example, the concentration changes over time can be further partitioned into compartments by time (separation based on frequency, which is different for heart and respiratory variations, for example), or by saturation (the total changes in blood volume and saturation can be analyzed as changes in multiple compartments (such as partition into a venous component of 70% saturation versus an arterial compartment of 98% saturation).

First, it should be understood that the compartment analysis (arterial vs. venous, or gravity vs. pulse) and the substance analysis (hemoglobin, fat, water, skin) can be performed simultaneously, and that they are performed sequentially here for the purposes of clarity of illustration. Further, the analysis can be processed in an iterative manner, which optimizes the separation based on different values of arterial and venous saturation, or upon different time constants for respiratory versus cardiac function.

Next, there are other methods that can be applied to this analysis. Time filtering, such as using a Fourier Transform to place the data into frequency-space from time-space, as is known in the art of data analysis, and can separate a regular heart rate from the pulse effects of respiration, as is shown in a later example.

All of these fall within the scope of the present exemplary technique if used in a multispectral or compartmental (or both) analysis to extract non-contact or loose-fit physiological parameters such as heart rate, respiratory rate, R-R heart beat interval, pulse oximetry, or tissue oximetry, cardiac function, bilirubin levels, sweat levels, hydration status, fat/water levels or ratios, cholesterol levels, or the like.

### EXAMPLE 11

### Rapid and Robust Determination of Rate from Intervals

Measurement of intervals, such as the interval time between peak arterial pulse timing, or the interval time between breaths, is an advantageous method to monitor rates in living subjects.

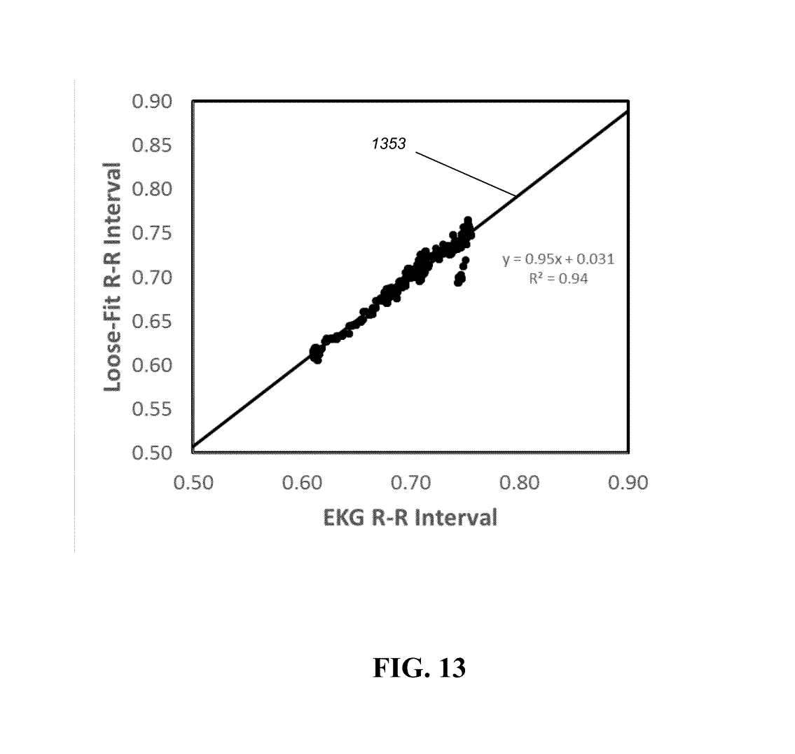

Interval measurement by optical methods correlates well with measurement of intervals via the gold-standard EKG, as shown in FIG. 13. Here, data from another human subject undergoing an exercise protocol and measured by both optical and electrical methods are shown. Plot line 1353 is the best-fit linear plot between the loose-fit arterial compartment beat-to-beat interval, and the electrode-based EKG beat-to-beat heart interval, both plotted in seconds. The plot is very nearly linear, with a correlation (r²) between both measures of 0.94, showing the measure is accurate during exercise. From each of these points, an estimated heart rate (in beats per minute) may be determined as 60/interval, where the interval is expressed in seconds. Use of intervals in order to determine rate allows for several advantages.

First, consider a heart rate of 115 beats per minute. This would be an interval of 0.52 seconds between each beat, and the heart rate could be estimated by 60/Tiₙₜₑᵣᵥₐₗ, where 60 is the number of seconds in a minute, Tᵢₙₜₑᵣᵥₐₗ is the beat-to-beat interval, and the result is in beats per minute.

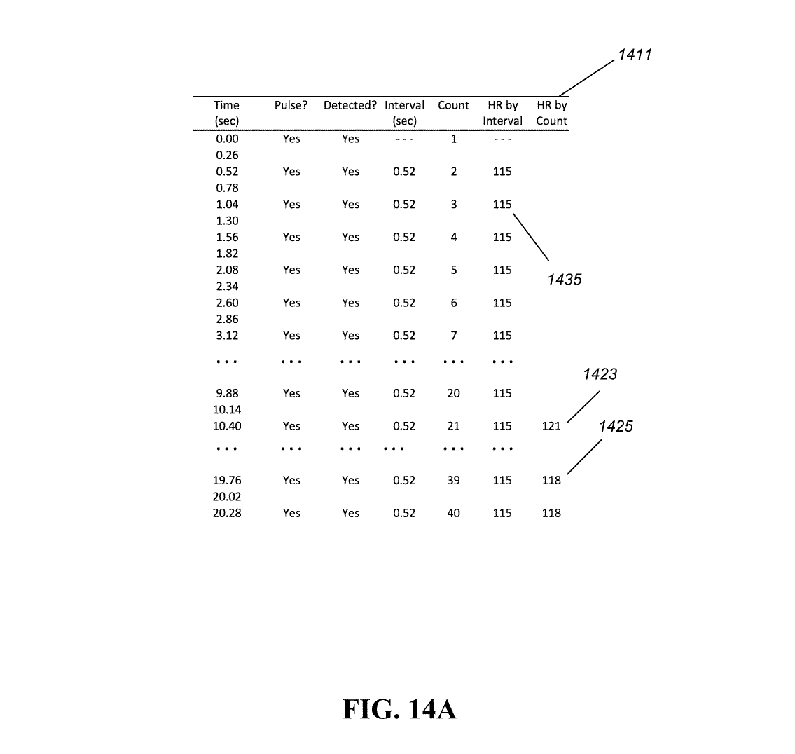

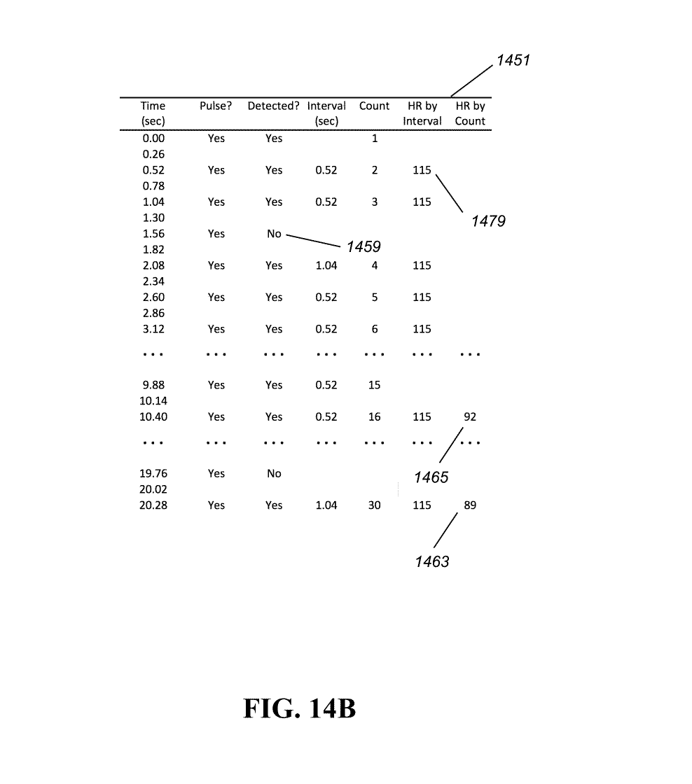

Data accumulates, as shown in FIG. 14A-B. In the case of FIG. 14A, the data are relatively noise free, while in the case of FIG. 14B the data are noisy with data dropouts. Both show model data for a heart rate of 115 beats/min.

In FIG. 14A, data are shown in table 1411. FIG. 14A shows rate estimation in the presence of good data with no drop-outs. FIG. 14B shows rate estimation in the presence of noise with some signal drop out. Referring back to FIG. 14A, after 1.00 seconds, 2 heart beats have been detected; by 10 seconds, 20 beats have been detected. To count to a stable number that estimates heart rate within a few beats per minute, perhaps 20 or 30 seconds would pass, at which time 40 to 60 beats would have been counted since the start. Here, a rate of 123/min is seen in the “HR by Count” column at data point 1423, while a rate of 117/min would be displayed (from multiplying the count of 39 times 60, and dividing by the counting period of 20 seconds) at data point 1425. In contrast, if an interval method is used, a heart rate of 115/min is seen in the “HR by Interval” column after 1 second has elapsed, at data point 1435, a time when heart rate by counting is blank. The count-based heart rate remains blank as the number of heartbeats (2 beats over 1 second) is insufficient to determine whether the heart rate is 90 (1.5 per second) or 150 (2.5 per second). This difficulty is made even worse if the signal is noisy, as it often is in real world measurements on mobile, active living subjects, as is discussed below. In some examples, the ability to determine a rate in 1 second using an interval method represents a significant improvement over counting.

First, the user can receive a heart rate estimate in as little 1-2 seconds or less. In contrast, a runner would have to wait 20 seconds to see the heart rate using a counting system. Anyone who has watched a runner pause for heart rate measurement, and grow impatient standing still, knows that this is significant user experience for athletes and other users.

Second, if the process of measurement requires power, such as driving an amplifier or illuminating an LED, a good heart rate could be determined by interval by having the watch on a few seconds each minute, as opposed to counting for much longer periods. The impact of this can be estimated. For a wristband with a small watch battery (such as the 25 mAh CR1216-type battery used in the Timex Indiglo, Timex, Connecticut), the difference between a 3 mA draw (for a typical LED) occurring 2 seconds each minute, versus having to stay on nearly constantly for good counting, is the difference between a 250 hour (10 1/2 day) battery life, and an 8 hour battery life.

Third, interval measures are surprisingly robust. Consider a runner with body movement that causes every 4^{th} heartbeat to be missed. This is shown in FIG. 14B. Here, At a rate of 115 beats/minute, the interval measured is first 0.52 sec, then 0.52 sec, but then 1.04 sec including the missed 4^{th} beat in table 1451 at data point 1459, then 0.5 sec again, 0.5 sec, and then 1.0 sec, and repeating this pattern.

By counting, 3 beats would be seen every 4 seconds, or 90 per minute, as shown by a count of 30 beats in 20 seconds at data point 1463 which is significantly in error, and worse, medically misleading.

In contrast, using the interval method, the modal (most frequent) interval would still be 0.5 sec, for an estimated and still-accurate heart rate estimate of 115 beats per minute at data point 1479. In fact, the 1.0 sec interval could easily be detected as being exactly twice the most frequent rate, and thus clearly determined to be a missed beat double interval. In contrast, the counting method would estimate the heart rate at approximately 90 beats/min regardless of the counting interval. An interval method is thus robust, especially one that uses modal or other filtering.

Of note, there are many ways to estimate intervals. For example, methods to detect cyclic rates such as Fourier transforms, wavelength analysis, and the like are well within the skills on one expert in signal processing.

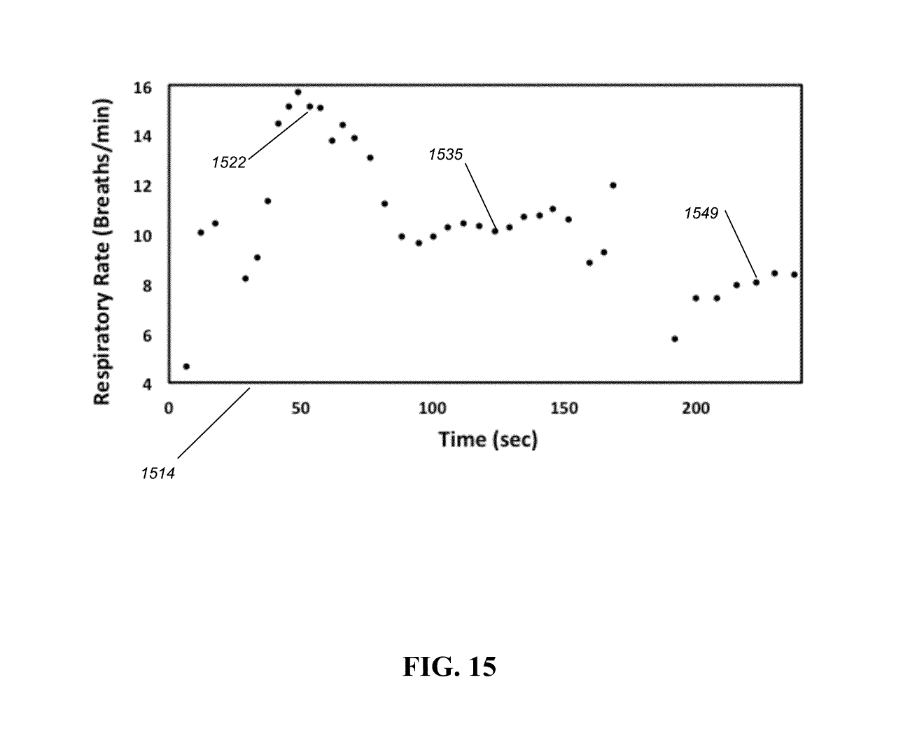

The interval method can be applied to respiratory rates as well. In FIG. 15, respiratory rates determined using an interval method are shown in graph 1514. In human studies, when the respiratory rate was controlled to be 15 breaths per minute, a rate of 15/min was determined by modal interval plotting, shown as time point 1522. When the respiratory rate was controlled to be 10 breaths per minute, a rate of 10/min was determined by modal interval plotting, shown as time point 1535. And last, when the respiratory rate was controlled to be 7.5 breaths per minute, a rate of 7 to 8/min was determined by modal interval plotting, shown as time point 1549.

### EXAMPLE 12

### Measurement of Calories Used

One of the features that can be measured using this approach is calories, either calories consumed or calories expended. In this example, it is determined in part based on a function of respiratory rate, as derived in the previous example.

Measuring calories consumed is a common laboratory experiment, and is typically performed using the relationship between the calories burned and the oxygen consumed. It is known that in the production of ATP, the energy currency of the eukaryotic cells that occurs in cells, and to a large extend near the mitochondria of the cell, that oxygen is consumed in an electron transfer called the electron transport chain, involving certain enzymes including cytochrome a/a3, cytochrome c, and others. Thus, the basis of calorie measurement in the laboratory is typically a measure of the amount of oxygen consumed, easily measured by flowing a controlled amount of oxygen into an exercise rebreathing setup that uses a closed breathing system.

It is an important realization that in this process, carbon dioxide is also produced. However, in laboratory systems, the carbon dioxide is often scrubbed away, such as by using alkaline agents that react with free carbon dioxide which the carbon dioxide reacts with. While typically ignored this carbon dioxide may become important later.

Another important realization is that the mammalian respiratory rate (at least as well studied in humans) is driven strongly by acidity of the blood and carbon dioxide levels. In contrast, oxygen does not drive respiration, save in certain end-stage lung disease. Humans placed in low oxygen airplanes at altitude may often lose consciousness before responding to their own low oxygen. Our realization includes that because reparatory rate is driven by carbon dioxide more than oxygen and carbon dioxide is produced in proportion to calories consumed, that the respiratory rate is related to calories. The final step is since we have demonstrated how to measure respiratory rate in a noninvasive, noncontact manner, that this measure can be used to estimate calorie consumption in an active, healthy person, such as during exercise using a wearable sensor.

Deriving a relationship between calories used and respiratory rate requires establishing multiple relationships. Some of these relationships have been determined, often for reasons having nothing to do with the real time monitoring of calorie consumption.

Layton (1993) developed new methodology for estimating breathing rates to determine doses resulting from exposure to airborne gases and particles. In this case, calories were not the goal of this research, but rather Layton was looking to develop scales for toxicity. Breathing rates were related to oxygen consumption associated with energy expenditures utilizing a ventilatory relationship that related minute volume to oxygen uptake as given by the equation V=E×H×VQ (where V is ventilation in L/day, E is energy expenditure in kcal/day, H is volume of oxygen consumed in the production of 1 kJ of energy in liters of oxygen/kcal, and VQ is the “ventilatory equivalent”). H is taken to be 0.21 liters of oxygen per kcal based on a 1977-1978 Nationwide Food Consumption Survey (USDA, 1984) and the NHANES II study (US DHHS 1983). VQ is taken to be 27 (unitless) representing the ratio of minute volume to oxygen uptake, a value is derived by Layton from published data of five researchers (Bachofen et al. 1973; Grimby et al. 1966; Lambersten et al. 1959; Saltin and Astrand 1967; Salzano et al. 1984). Layton's equation was later supported by the OEHHA Report (2000).

We want to estimate calories based on respiratory rate. To begin, we modified Layton's equation for our purposes to solve instead for energy expenditure in kcal/min, instead of solving for minute ventilation, as: E=V/(H×VO). By doing this we asking a different question from the investigators interested in calculating respiratory exposure. However, the relationship between minute ventilation and respiratory rate was not clear.

To estimate minute ventilation given a respiratory rate measured by the device, we modified the work of Naranjo et al. (2005) who demonstrated a curvilinear relation between respiratory rate and minute volume expressed by an exponential function. This study recruited trained athletes and tested them on two different treadmill protocols. Expired air was collected and analyzed for carbon dioxide and oxygen, as well as liter flow. From this they determined one relationship between tidal volume, inspiratory and expiratory duration, and respiratory rate. A nomogram was developed for a relation between tidal volume (y) and respiratory rate (x) in this group of trained athletes, with a split by phenotypic gender: y=9.6446 e^{0.9328x} for women, and y=8.3465 e^{0.7458x} for men.

The work of Naranjo addresses breathing patterns in one group of subjects, but makes no association with calories consumed and the approach fails for subjects breathing at low rates and in non-exercise conditions.

We modified Naranjo's relationships to derive new functions to estimate energy expenditure (in kcal/min) from respiratory rate (in breaths/min) for both men and women. In one example, this relationship was best represented by second-order polynomial equations where the minimum values are the predicted resting metabolic rate, as follows: y=0.0044x²+0.0798x−0.2106 for women (r²=0.998) and y=0.0069x²+0.0463x−0.0324 for men (r²=0.999). The ability to accurately, non-invasively quantify respiratory rate allows us to combine disparate research to develop a novel solution to measuring metabolism in real-time.

Using these equations, we can now display real-time estimates of calories consumed, using the respiratory rates determined using the method of the previous example, and the calorie conversions as determined in this example.

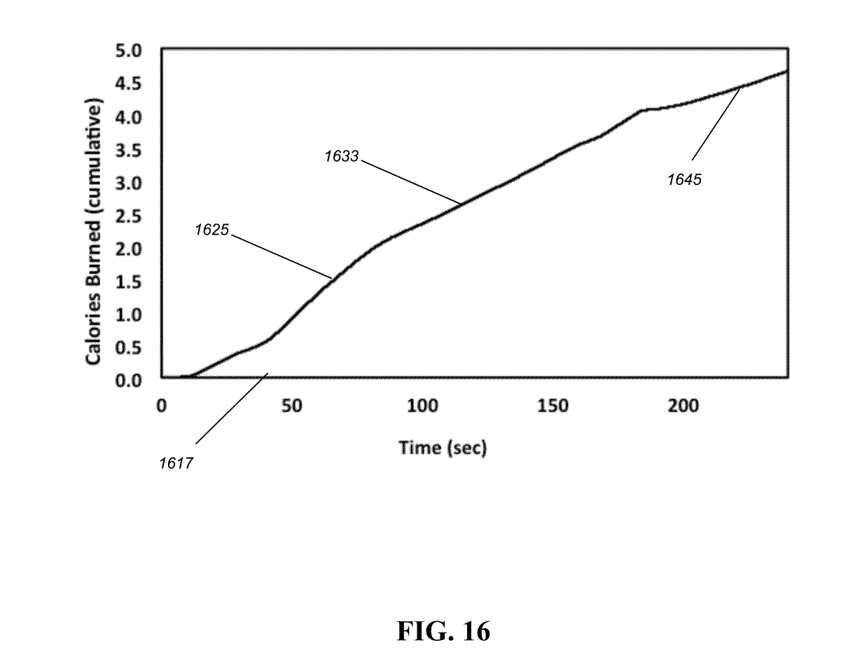

Results from a human subject are shown in FIG. 16. Here, cumulative calories were calculated, and could be displayed in real time on a wearable watch. A plot of one subject's data is shown as graph 1617. At time point 1623, the subject is breathing more quickly, and this is reflected in a more rapid increase in calories expended, as shown at time point 1625. As the breathing is slowed, there is slower accumulation at time point 1633. Last, at the slowest respiratory rate, the accumulation is slower still at time point 1645.

Accelerometers may be incorporated into a an implementation of the described techniques, in some examples, to provide additional and/or supplemental data to optical respiration measures within the spirit of the present exemplary technique provided that noninvasive and/or noncontact respiratory signals are incorporated into the analysis.

Second, in additional contrast, some other known devices for estimating calories use global positioning (GPS) signals and map data to calculate a distance traveled over time, (e.g., Runtastic, San Francisco, Calif.) and also input such as mode of movement (walking, running, skating, cycling, etc.) in order to estimate calories used. Such GPS and map data could be incorporated into the present device to provide additional data to the optical respiration measures within the spirit of the present exemplary technique provided that noninvasive and/or noncontact respiratory signals are incorporated into the analysis.

Third, a respiratory measure is a robust measure of calories. When working at high effort, our respiratory rate naturally rises to provide the ventilation used. But such a high rate is difficult to “fake”. If a high rate of breathing is attempted when at rest, the carbon dioxide levels in the bloodstream may rapidly fall away from normal values, resulting in alkaline blood, changes in brain blood flow, lightheadedness, and even loss of consciousness.

### EXAMPLE 13

### Measurement of Calories Consumed and Calorie Balance

In addition to calories used or expended, the number of calories ingested are an important part of the equation. Here, the calculations of Example 4 are relevant. Fat has an absorbance peak at multiple points, including local peaks at 760 nm, 920 nm, and elsewhere. By detecting changes in the peaks of the fat levels, and integrating over time, a measure of the fat calories consumed can be estimated. One exemplary method would be to then assume that fat comprises a fixed amount of dietary calories, and total calories ingested can be estimated as Intake (in kcal or kJ)=Cᵢₙ/F_{fat}, where Cᵢₙ is the estimated total calories ingested, and F_{fat} is the fraction of calories estimated to come from fat.

Once calories used and calories ingested are calculated, a calorie balance over the day can be determined as: C_{bal}=Cᵢₙ−C_{used}, where C_{bal} is the calorie balance over a period of time, Cᵢₙ is the estimated total calories ingested, and C_{used} is the estimated total calories used. In this way, a user could adjust the calories consumed by eating and drinking to balance the calories burned or used during the day.

### EXAMPLE 14

### Measurement of Hydration

In addition to calorie balance, other balances are important to a user. For example, the water balance could be calculated. Again, using the calculations of Example 4, water concentrations can be calculated. Here, water has absorbance peaks at multiple points, including local peaks near 960 nm and elsewhere, and second differential peaks near 820 nm. By detecting changes in the peaks of the water levels over time, a measure of the hydration of the subject may be determined.

For example, dehydration may lower the water content at the skin, in the tissues, result in a higher hemoglobin concentration in the blood and capillaries, and reduce the perfusion of the capillaries. In contrast, a drink of water or fluids would, when absorbed, result in the opposite: an increase in the sweat water content at the skin, an increase in the water in the tissues and capillaries, and a drop in hemoglobin concentration in the blood and capillaries, increases in perfusion of the capillaries.

A time since last hydration can be determined, and an automated detection of intake can be determined. One exemplary method would be to then assume that fat comprises a fixed amount of dietary calories, and total calories ingested can be estimated as Intake (in kcal or kJ)=Cᵢₙ/F_{fat}, where Cᵢₙ is the estimated total calories ingested, and F_{fat} is the fraction of calories estimated to come from fat.

### EXAMPLE 15

### Ambient Light

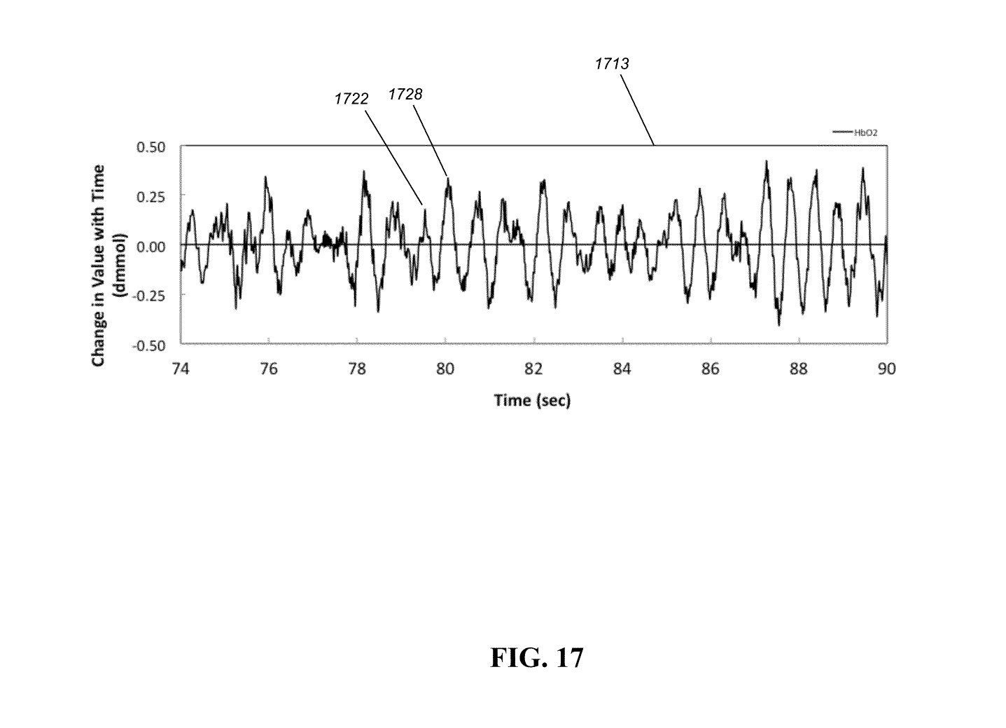

As an example, the heart rate pulse is shown from a signal collected in ambient light in FIG. 17 shows an exemplary multispectral signal detected using ambient light; and

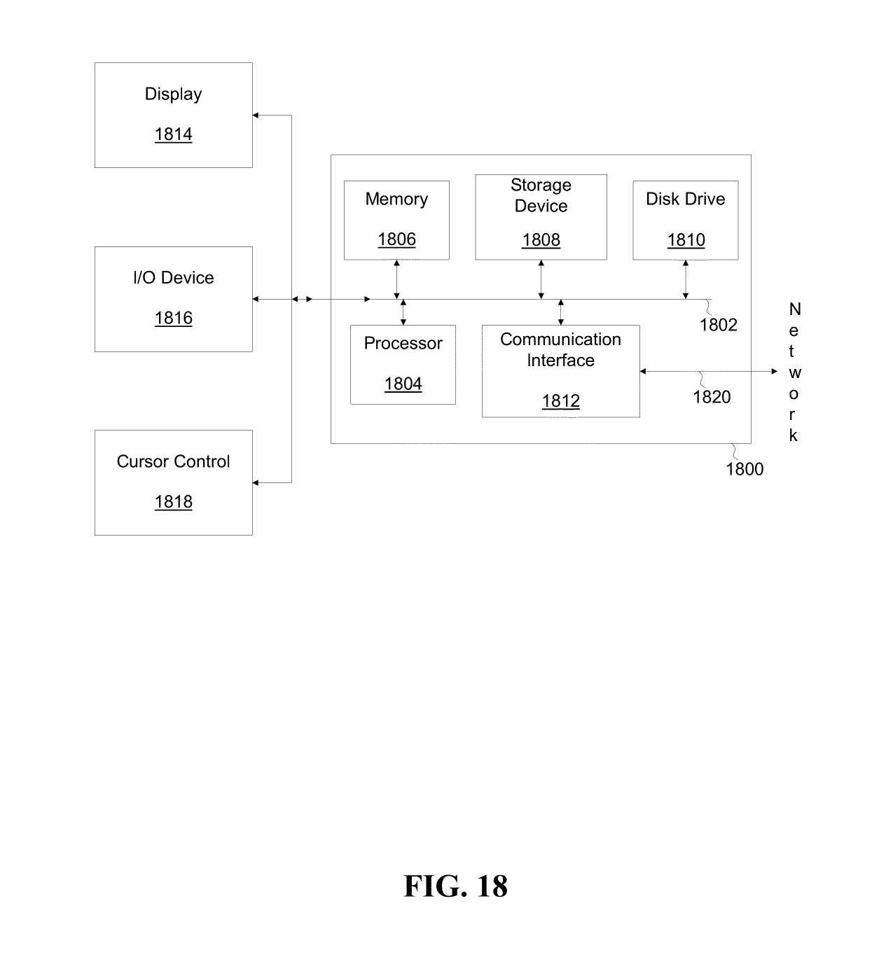

FIG. 18 is a block diagram illustrating an exemplary computer system suitable for rapid rate respiration determination. Data were collected at a distance. The signal is clearly visible as pulse peaks (for example, pulse peaks 1722 and 1728 where collected from distance of 10 cm from the subject in ambient light. Such signals can be processed as described in earlier examples, and as taught related disclosures. Depending on the number of wavebands selected, and their range, such signals can be used to extract heart rate, respiratory rate, heart rate variability, respiratory rate, calories, hydration, sleep state (based on rate and variability), even blood alcohol or blood fat levels.

### EXAMPLE 16

### Sleep Stage

Many sleep-stage bands collect accelerometer data. Such devices determine sleep stage by motion, which can be very inaccurate. In contrast, heart rate, heart rate variability, and respiratory rate also fit into these equations. Once a good measure of heart rate, heart rate variability, and respiratory rate is obtained using the methods described herein, sleep stage can be extracted using the equations and methods from the published literature. More accurately, a database can be assembled using remote monitoring from the optical devices disclosed herein.

FIG. 18 is a block diagram illustrating an exemplary computer system suitable for rapid rate respiration determination. In some examples, computer system 1800 may be used to implement computer programs, applications, methods, or other software to perform the above-described techniques such as those described above. Computer system 1800 includes a bus 1802 or other communication mechanism for communicating information, which interconnects subsystems and devices, such as processor 1804, system memory 1806 (e.g., RAM, or the like), storage device 1808 (e.g., ROM, or the like), disk drive 1810 (e.g., magnetic, optical, or the like), communication interface 1812 (e.g., modem, Ethernet card, or the like), display 1814 (e.g., CRT, LCD, or the like), input device 1816 (e.g., keyboard, or others), and cursor control 1818 (e.g., mouse, trackball, or the like).

In some examples, computer system 1800 performs specific operations by processor 1804 executing one or more sequences of one or more instructions stored in system memory 1806. Such instructions may be read into system memory 1806 from another computer readable medium, such as static storage device 1808 or disk drive 1810. In other examples, hard-wired circuitry may be used in place of or in combination with software instructions to implement the exemplary techniques.

The term “computer readable medium” refers to any medium that participates in providing instructions to processor 1804 for execution. Such a medium may take many forms, including but not limited to, non-volatile media, volatile media, and transmission media. Non-volatile media includes, for example, optical or magnetic disks, such as disk drive 1810. Volatile media includes dynamic memory, such as system memory 1806. Transmission media includes coaxial cables, copper wire, and fiber optics, including wires that comprise bus 1802. Transmission media can also take the form of acoustic or light waves, such as those generated during radio wave and infrared data communications.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, carrier wave, or any other medium from which a computer can read.

In some examples, execution of the sequences of instructions may be performed by a single computer system 1800. Two or more computer systems 1800 coupled by communication link 1820 (e.g., LAN, PSTN, wireless network, or the like) may perform the sequence of instructions in coordination with one another. Computer system 1800 may transmit and receive messages, data, and instructions, including program (i.e., application code) through communication link 1820 and communication interface 1812. Received program code may be executed by processor 1804 as it is received, and/or stored in disk drive 1810, or other non-volatile storage for later execution. In other examples, the above-described computer, computer system, data processing techniques, or elements, such as those described, may be varied in form, function, feature, feature set, implementation, or other details without limitation to the specific examples provided.

In some examples, smart bio-aware sensors may have multiple expected and unexpected advantages can result from using broadband white LED illuminators and spectrally-resolved detectors in mobile devices, especially when combined with integrated processing power. In certain applications, such as fitness applications, this improvement may occur without undue space and size constraints and without degrading or with improvement in output stability. We show that bio-aware sensors can be achieved by (a) using broadband light, from the room or from a white LED source, which results in low energy consumption, and (b) using the sensor built into the phone or watch, with spectral sensitivity added, such that the improved sensor can even be embedded into bracelets, pendants, and phones. Bio-awareness simplifies measurements (by allowing for quantitative detection of gestures and physiology), and improves data quality. Such improved sensors may permit a light source and detector to be embedded into nearly any mobile device, such as into a smartphone, bracelet, pendant, shoe, clothing, or watch.

In other examples, an improved calorie sensor for mobile use is disclosed, including an illuminator sensor phone and watch have been constructed and tested, in which a phosphor-coated white LED and sensor have been configured so as to allow spectroscopic filtering, to produce (if needed in the absence of adequate ambient light or to replace ambient light) a continuous, broadband light from 400 nm to 700 nm, and a spectrally resolved detection. The resulting sensor is sensitive to physiology (heart rate, respiratory rate, calories, hydration), as well as to type and state (finger, hand, live, dead), for analysis and initiating actions based on the result. This device has been built and tested in several configurations in models, animals, and humans, and has immediate application to several important problems, both medical and industrial, and thus constitutes an important advance in the art.

None of the above systems suggest or teach a method or system exhibiting determination of a repetitive cycle in half to 2 cycles, or less. Such methods include monitoring cycle portions of known length relative to rate, or looking at cycle intervals, to rapidly determine a cardiac or respiratory rate.

Although the foregoing examples have been described in some detail for purposes of clarity of understanding, the above-described inventive techniques are not limited to the details provided. There are many alternative ways of implementing the above-described exemplary techniques. The disclosed examples are illustrative and not restrictive.

We claim:

1. A device, comprising:
a sensor configured to detect light, and
a processor with a machine-readable hardware code, said code when executed by the processor generating, based upon said detected light, an output measure that is a function of an estimated rate of at least one repetitive event, said output based at least in part on one or more intervals of time elapsed between occurrences of said at least one said repetitive event.

2. The device of claim 1, wherein the light detected is backscattered from a subject.

3. The device of claim 1, wherein the light detected is transmitted through a subject.

4. A device, comprising:
a sensor configured to detect broadband ambient light, the sensor having a filter configured to produce a sensor region, the sensor region configured to sense a predetermined waveband of the broadband ambient light detected; and
a processor being configured to process an instruction, the instruction, when executed by the processor, generating, based upon the detected light, an output measure that is a function of an estimated rate of a repetitive event, the output measure being based at least in part on a time interval elapsed between an occurrence of the repetitive and another repetitive event.

5. The device of claim 4, wherein the broadband ambient light detected is backscattered from a subject.

6. The device of claim 4, wherein the broadband ambient light detected is transmitted through a subject.

7. A device, comprising:
a sensor configured to detect broadband ambient light, the sensor having a filter configured to produce a sensor region configured to be sensitive to a predetermined waveband of the broadband ambient light;
a processor having one or more program instructions that, when executed by the processor and based upon the broadband ambient light detected by the sensor, is configured to perform a waveform analysis of the broadband ambient light to determine a timing associated with a repetitive event using the broadband ambient light detected by the sensor; and
generating an output measure that is a function of an estimated rate of the repetitive event and another repetitive event, the output measure being based on the waveform analysis.

8. The device of claim 7, wherein the broadband ambient light is backscattered from a subject.

9. The device of claim 7, wherein the broadband ambient light is transmitted through a subject.

10. The device of claim 7, wherein the device is a mobile health monitor.

11. The device of claim 7, wherein the device is a mobile phone.

12. The device of claim 7, wherein the device is a wearable device.

13. The device of claim 7, wherein the device is wearable clothing.

14. The device of claim 7, wherein the device is a wearable glasses.

15. The device of claim 7, wherein the device is a wearable bracelet.

16. The device of claim 7, wherein the device is a wearable earphone.

17. The device of claim 7, wherein the device is a wearable contact lens.

18. The device of claim 7, wherein the device is a security system, a room occupancy sensor.

19. The device of claim 7, wherein the device is a room occupancy sensor.

20. The device of claim 7, further comprising a spectral filter deposited directly on the sensor, wherein the sensitivity of the detector to a wavelength band uses a filter.

21. The device of claim 7, wherein the sensor is configured to operate in a non-contact manner with the subject.

22. A device, comprising:
a sensor configured to detect broadband ambient light after the ambient light is transmitted through a subject, the sensor having a filter configured to produce a sensor region configured to be sensitive to a predetermined waveband of the broadband ambient light detected;
a processor having one or more program instructions that, when executed by the processor and based upon the broadband ambient light detected by the sensor, performing a waveform analysis of the broadband ambient light to determine a timing or frequency associated with at least one repetitive event using the broadband ambient light detected by the sensor; and
generating an output measure that is a function of an estimated rate of the repetitive event and a subsequent repetitive event, the output being based on the waveform analysis.
